(19) United States
(12) Patent Application Publication
(10) Pub. No.: US 2025/0303062 A1
(43) Pub. Date: Oct. 2, 2025

(54) Methods for controlling drug administration

(71) Applicant: NUTROMICS TECHNOLOGY PTY LTD, Brunswick (AU)

(72) Inventors: Agim Beshiri, Brunswick (AU); Vinicius Goncales, Brunswick (AU); Jeremy Van Eps, Brunswick (AU); Mikel Fraser, Brunswick (AU); Garry Chambers, Brunswick (AU)

(21) Appl. No.: 19/234,956

(22) Filed: Jun. 11, 2025

Related U.S. Application Data
(63) Continuation of application No. PCT/AU2023/051315, filed on Dec. 16, 2023.
(60) Provisional application No. 63/433,145, filed on Dec. 16, 2022.
(60) Provisional application No. 63/433,151, filed on Dec. 16, 2022.
(60) Provisional application No. 63/433,158, filed on Dec. 16, 2022.
(60) Provisional application No. 63/433,164, filed on Dec. 16, 2022.

(51) Int. Cl. A61M 5/172; A61K 9/00

(52) U.S. Cl. CPC A61M 5/172; A61K 9/0009; A61M 2205/3303

(57) ABSTRACT

Provided is computer-implemented method for administering a drug to a subject. The method includes administering the drug to the subject using a controllable pump, contacting a fluid of the subject, such as interstitial fluid in situ, with an electrochemical aptamer-based sensor capable of detecting the drug, receiving a series of output values of the electrochemical aptamer-based sensor over a period of time, and using the series of output values to control the pump.

### CROSS-REFERENCE TO RELATED APPLICATIONS

The present application is a Bypass Continuation Application of International Application No. PCT/AU2023/051315, filed Dec. 16, 2023, and published as WO 2024/124305 A1 on Jun. 20, 2024, in English, which claims priority from U.S. provisional patent application 63/433,145, filed Dec. 16, 2022; U.S. provisional patent application 63/433,151, filed Dec. 16, 2022; U.S. provisional patent application 63/433,158, filed Dec. 16, 2022; and U.S. provisional patent application 63/433,164, filed Dec. 16, 2022; the contents of which are hereby incorporated by reference in their entireties.

### FIELD OF THE INVENTION

The present invention relates generally to the control of a drug in the body of a subject over time. Such control allows for maintenance of the plasma concentration of the drug within a therapeutic window.

### BACKGROUND TO THE INVENTION

It is well accepted that valuable information may be obtained from monitoring the amount of a drug in the body of a subject over time. In a healthcare facility (such as a hospital), monitoring is typically performed by a clinician ordering a drug assay with one or more other staff members of the healthcare facility obtaining a sample of blood from the subject at regular time intervals, sending each collected sample to a laboratory for analysis, where the analysis of each assay is performed, and the result of each assay is communicated by some means to the clinician. In this way, the clinician is able to monitor the level of the drug as it rises, falls, or remains steady, over a time period. The information provided may allow the clinician to better manage a condition of the subject by improved pharmacotherapy.

Modern medicine provides clinicians with a broad range of drugs at their disposal for treating or preventing disease states. While pharmacokinetic and efficacy data will often be available for a drug, such data may be of limited applicability for a clinician seeking to optimise dosage for a given subject or circumstance. It is a well understood in the art that significant inter-subject variability exists with regard to the rate and extent of transport of a drug to a relevant tissue or organ. Clinically important differences in the rates of clearance and metabolism of a drug are also noted. Such variability may arise from factors such as genetics, sex, age, ethnicity, hydration state, and comorbidities, for example.

In light thereof, methods for monitoring drugs have been implemented to determine (as a function of time) the amount of drug in the plasma. The data output by such methods guides a clinician in devising dosage regimens relevant to a subject under treatment so that drug concentrations can be maintained within a therapeutic range, having regard to any toxicity. Well-resourced healthcare facilities, such as hospitals, typically provide services which provide support for drug monitoring and interpretation of results.

Drug monitoring is typically more useful where a drug is used to prevent an adverse outcome such as graft rejection or to avoid toxicity, as with aminoglycosides. A drug may satisfy certain criteria to be suitable for drug monitoring. Examples include narrow target range, significant pharmacokinetic variability, a reasonable relationship between plasma concentrations and clinical effects, established target concentration range, and availability of a reasonably accurate drug assay. More commonly monitored drugs include carbamazepine, valproate, digoxin, and vancomycin.

For some drugs, monitoring is used to assist diagnosis (e.g., salicylates).

Drug monitoring typically involves measuring drug concentrations in plasma or serum over a monitoring period commencing around the time of administration. Problems arise in that the process of taking blood may be uncomfortable for the subject, and time consuming for the relevant hospital personnel. Furthermore, each sample must be assayed for the relevant drug, a process which is resource-intensive and, even when performed urgently, provides data that is far from reflective of the subject's current state.

Generally, only a small number of samples are taken over a monitoring period. While such a limited amount of data is of some use to the clinician, important pharmacokinetic features such as peak concentration (Cₘₐₓ) and time to peak concentration (Tₘₐₓ) are typically missed. Other parameters such as total drug exposure (as determined by area under the curve, “AUC”) and elimination half-life may be inaccurate.

The prior art provides various means to address the problems discussed above. For example, Bayesian methods may be used to assist in dosage decisions. Such methods may be used in attempts to predict pharmacokinetic values, dosage regimens, and serum concentrations for drugs. Bayesian methods rely on population-based pharmacokinetic parameters, which are applied to a small number of observed serum concentrations in the subject. While these methods provide some useful output, they generally fail to provide reliable means for maintaining the plasma concentration of a drug within a therapeutic window.

A further problem with drug monitoring is that timing of samples taken from a subject is critical in obtaining accurate information. It is not uncommon for the time reported for sample collection to be very different to the actual time the sample was obtained. For example, a nurse may take a blood sample for drug monitoring at 10:15 am but may delay entering the time in the subject's records, often due to their attention being directed to another urgent task. When the time is entered, the nurse may record the time of entry (which is later than the time of collection) or alternatively estimate the time of collection as earlier or later than the actual time. Such inaccuracies may significantly confound interpretation of monitoring data leading to adverse clinical outcomes, especially where the drug has a narrow target concentration range such as vancomycin.

Where the time recorded is later than actual collection, the real concentration of a drug at that later time may be lower and in which case an erroneously low dosage (and possibly a dosage having unacceptably low efficacy) may be administered at the next dosage time point. Conversely, where the time recorded is earlier than actual collection, the real drug concentration at that later time may be lower and in which case an erroneously high dosage (and possibly a dosage leading to unacceptable toxicity) may be administered.

Even where a time of collection is accurately recorded, the sampling protocol may fail to allow for accurate control of a drug within a range. Reference is made to FIG. 1A showing a graph of drug serum concentration versus time. The dark boxes show the times at which serum is sampled and assayed for drug. As will be appreciated, a clinician considering the second data point can see that the drug is approaching the toxic levels and adjust the rate of infusion downwardly. However, the data did not become available until one hour after the blood was drawn, and in that time the drug concentration has crossed into the toxic region. In a similar manner, a clinician considering the fourth data point will note the drug has fallen to its minimum efficacious concentration, and therefore adjust dosage upwardly. In the time it has taken to perform the relevant assay, the drug has fallen well below its minimum efficacious concentration as shown on the graph. Even where a greater number of data points are provided, the delay in obtaining assay results from the laboratory may prevent timely adjustment of a dosage regimen, resulting in excursions from the therapeutic window for the drug concerned.

A further problem with the prior art is the delay between a drug assay result and the drug dosage adjustment required in light of the result. It is often the case that due to high workload or other distractions hospital personnel cannot immediately action any required adjustment to drug dosage. Such delay may allow a drug concentration to travel outside the therapeutic window.

Yet a further problem in prior art dosing methods is that drug concentration may be modulated over time between an upper and lower limit. The amplitude of the modulation may be relatively high, meaning that for a large proportion of the treatment time the drug is at a sub-optimal level. For example, while drug levels may be maintained above a minimum inhibitory concentration with prior art methods, concentrations higher than the minimum inhibitory concentration (and possibly very near a toxic concentration) will have a far greater efficacy leading to superior clearance of infection. Given the toxicity problems of some drugs, such as vancomycin, it is not possible with prior art method to maintain drug level just below the toxic level given the danger that concentration of the drug will exceed the toxic concentration for periods of time. Put a different way, fine control over drug concentration is not possible with methods of the prior art.

It is an aspect of the present invention to provide an improvement in prior art methods and/or systems for monitoring a drug in a subject. It is a further aspect of the present invention to provide a useful alternative to prior art methods and/or systems for monitoring a drug in the body of a subject.

The discussion of documents, acts, materials, devices, articles, and the like, is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

In a first aspect, but not necessarily the broadest aspect, the present invention provides a computer-implemented method for administering a drug to a subject, the method comprising:
administering the drug to the subject using a processor-controllable agent administration apparatus,
contacting a fluid of the subject with an electrochemical aptamer-based sensor capable of detecting the drug,
receiving a series of output values or derivatives thereof of the electrochemical aptamer-based sensor over a period of time, and
using the series of output values or derivatives thereof to control the processor-controllable agent administration apparatus,
wherein the output values or derivatives thereof are received at a time interval or an averaged time interval of less than about 4 hours, 3 hours, 2 hours, or 1 hour.

In one embodiment of the first aspect, the time interval or averaged time interval is less than about 50 minutes, 40 minutes, 30 minutes, 20 minutes, 10 minutes, 5 minutes, 4 minutes, 3 minutes, 2 minutes, 1 minute, 50 seconds, 40 seconds, 30 seconds, 20 seconds, 10 seconds, 9 seconds, 8 seconds, 7 seconds, 6 seconds, 5 seconds, 4 seconds, 3 seconds, 2 seconds, 1 second, 900 milliseconds, 800 milliseconds, 700 milliseconds, 600 milliseconds, 500 milliseconds, 400 milliseconds, 300 milliseconds, 200 milliseconds, 100 milliseconds, 90 milliseconds, 80 milliseconds, 70 milliseconds, 60 milliseconds, 50 milliseconds, 40 milliseconds, 30 milliseconds, 20 milliseconds, 10 milliseconds, 9 milliseconds, 8 milliseconds, 7 milliseconds, 6 milliseconds, 5 milliseconds, 4 milliseconds, 3 milliseconds, 2 milliseconds, or 1 millisecond.

In one embodiment of the first aspect, the drug is administered continuously or semi-continuously.

In one embodiment of the first aspect, the processor-controllable agent administration apparatus is a processor-controllable pump.

In one embodiment of the first aspect, the series of output values or derivatives thereof of the electrochemical aptamer-based sensor are used to generate a series of drug concentration values, and the processor-controllable agent delivery apparatus is controlled by reference to the series of drug concentration values or derivatives thereof.

In one embodiment of the first aspect, the series of drug concentration values or derivatives thereof indicate that the subject has or may be about to receive a maximum or supra-maximum amount of the drug, the processor-controllable agent delivery apparatus is controlled so as to cease or reduce a rate of administration of the drug.

In one embodiment of the first aspect, indication of the maximum or supra-maximum amount is determined by reference to a concentration value for the drug as determined by a concentration value of the series of drug concentration values or derivatives thereof.

In one embodiment of the first aspect, indication of the maximum or supra-maximum amount is determined by reference to an exposure for the drug over a period of time, the exposure determined by reference to the series of drug concentration values or derivatives thereof.

In one embodiment of the first aspect, the exposure is determined by reference to a curve of concentration of the drug over the period of time, the curve generated by reference to the series of output values or derivatives thereof of the electrochemical aptamer-based sensor.

In one embodiment of the first aspect, exposure is determined by determining the area under the curve.

In one embodiment of the first aspect, the maximum amount is a toxic amount, a potentially toxic amount, or a wasteful amount.

In one embodiment of the first aspect, where the series of drug concentration values or derivatives thereof indicate that the subject has or may be about to receive a minimum or sub-minimum amount of the drug, the processor-controllable agent delivery apparatus is controlled so as to commence or increase a rate of administration of the drug.

In one embodiment of the first aspect, indication of the minimum or sub-minimum amount is determined by reference to a concentration value for the drug as determined by a concentration value of the series of drug concentration values or derivatives thereof.

In one embodiment of the first aspect, indication of the minimum or sub-minimum amount is determined by reference to an exposure for the drug over a period of time, the exposure determined by reference to the series of drug concentration values or derivatives thereof.

In one embodiment of the first aspect, exposure is determined by reference to a curve of concentration of the drug over the period of time, the curve generated by reference to the series of output values or derivatives thereof of the EAAB sensor.

In one embodiment of the first aspect, exposure is determined by determining the area under the curve.

In one embodiment of the first aspect, the minimum amount is a minimum therapeutically or prophylactically effective amount.

In one embodiment of the first aspect, indication that the subject has or may be about to receive a minimum or sub-minimum or maximum or supra-maximum amount of the drug is determined by reference to a kinetic parameter of the drug in the fluid.

In one embodiment of the first aspect, the kinetic parameter is a rate of increase or decrease in the amount of the drug.

In one embodiment of the first aspect, the series of output values or derivatives thereof of the electrochemical aptamer-based sensor are used to control the processor-controllable agent administration apparatus to maintain the amount of drug within a predetermined range.

In one embodiment of the first aspect, the predetermined range is a therapeutic window or a prophylactic window.

In one embodiment of the first aspect, the amount of drug is considered in terms of a concentration of the drug in the fluid or exposure of the drug over a period of time.

In a second aspect, the present invention provides an apparatus for controlled administration a drug to a subject, the apparatus comprising:
an electrochemical aptamer-based sensor configured to contact a fluid of the subject, and
a processor in operable communication with the electrochemical aptamer-based sensor and having access to processor-executable instructions configuring the processor to:
receive a series of output values or derivatives thereof of the electrochemical aptamer-based sensor over a period of time, and
the processor and/or other processor(s) having access to the processor-executable instructions using the series of output values or derivatives thereof to predict the pharmacokinetic feature,
wherein the output values or derivatives thereof are received at a time interval or an averaged time interval of less than about 4 hours, 3 hours, 2 hours, or 1 hour.

In one embodiment of the second aspect, the time interval or averaged time interval is less than about 50 minutes, 40 minutes, 30 minutes, 20 minutes, 10 minutes, 5 minutes, 4 minutes, 3 minutes, 2 minutes, 1 minute, 50 seconds, 40 seconds, 30 seconds, 20 seconds, 10 seconds, 9 seconds, 8 seconds, 7 seconds, 6 seconds, 5 seconds, 4 seconds, 3 seconds, 2 seconds, 1 second, 900 milliseconds, 800 milliseconds, 700 milliseconds, 600 milliseconds, 500 milliseconds, 400 milliseconds, 300 milliseconds, 200 milliseconds, 100 milliseconds, 90 milliseconds, 80 milliseconds, 70 milliseconds, 60 milliseconds, 50 milliseconds, 40 milliseconds, 30 milliseconds, 20 milliseconds, 10 milliseconds, 9 milliseconds, 8 milliseconds, 7 milliseconds, 6 milliseconds, 5 milliseconds, 4 milliseconds, 3 milliseconds, 2 milliseconds, or 1 millisecond.

In one embodiment of the second aspect, the drug is administered continuously or semi-continuously.

In one embodiment of the second aspect, the processor-controllable agent administration apparatus is a processor-controllable pump.

In one embodiment of the second aspect, the series of output values or derivatives thereof of the electrochemical aptamer-based sensor are used to generate a series of drug concentration values or derivatives thereof, and the processor-controllable agent delivery apparatus is controlled by reference to the series of drug concentration values or derivatives thereof.

In one embodiment of the second aspect, where the series of drug concentration values or derivatives thereof indicate that the subject has or may be about to receive a maximum or supra-maximum amount of the drug, the processor-controllable agent delivery apparatus is controlled so as to cease or reduce a rate of administration of the drug.

In one embodiment of the second aspect, indication of the maximum or supra-maximum amount is determined by reference to a concentration value for the drug as determined by a concentration value of the series of drug concentration values or derivatives thereof.

In one embodiment of the second aspect, indication of the maximum or supra-maximum amount is determined by reference to an exposure for the drug over a period of time, the exposure determined by reference to the series of drug concentration values or derivatives thereof.

In one embodiment of the second aspect, exposure is determined by reference to a curve of concentration of the drug over the period of time, the curve generated by reference to the series of output values or derivatives thereof of the electrochemical aptamer-based sensor.

In one embodiment of the second aspect, exposure is determined by determining the area under the curve.

In one embodiment of the second aspect, the maximum amount is a toxic amount, a potentially toxic amount, or a wasteful amount.

In one embodiment of the second aspect, where the series of drug concentration values or derivatives thereof indicate that the subject has or may be about to receive a minimum or sub-minimum amount of the drug, the processor-controllable agent delivery apparatus is controlled so as to commence or increase a rate of administration of the drug.

In one embodiment of the second aspect, indication of the minimum or sub-minimum amount is determined by reference to a concentration value for the drug as determined by a concentration value of the series of drug concentration values or derivatives thereof.

In one embodiment of the second aspect, indication of the minimum or sub-minimum amount is determined by reference to an exposure for the drug over a period of time, the exposure determined by reference to the series of drug concentration values or derivatives thereof.

In one embodiment of the second aspect, exposure is determined by reference to a curve of concentration of the drug over the period of time, the curve generated by reference to the series of output values or derivatives thereof of the electrochemical aptamer-based sensor.

In one embodiment of the second aspect, exposure is determined by determining the area under the curve.

In one embodiment of the second aspect, the minimum amount is a minimum therapeutically or prophylactically effective amount.

In one embodiment of the second aspect, indication that the subject has or may be about to receive a minimum or sub-minimum or maximum or supra-maximum amount of the drug is determined by reference to a kinetic parameter of the drug in the fluid.

In one embodiment of the second aspect, the kinetic parameter is a rate of increase or decrease in the amount of the drug.

In one embodiment of the second aspect, the series of output values or derivatives thereof of the electrochemical aptamer-based sensor are used to control the processor-controllable agent administration apparatus to maintain the amount of drug within a predetermined range.

In one embodiment of the second aspect, the predetermined range is a therapeutic window or a prophylactic window.

In one embodiment of the second aspect, the amount of drug is considered in terms of a concentration of the drug in the fluid or exposure of the drug over a period of time.

### BRIEF DESCRIPTION OF THE FIGURES

FIG. 1A is a graph of drug concentration as a function of time after an initial administration of the drug at t=0, showing the times at which bloods samples are taken for drug assay. Repeated dosages of drug are given with the aim of maintaining concentration within a therapeutic window as indicated by the dashed lines. Excursions beyond the therapeutic range will be noted.

FIG. 1B is a graph of drug concentration as a function over time resulting from dosage control according to the present invention. Maintenance of drug concentration within the therapeutic range will be noted.

FIG. 2 illustrates in graphical form data showing the response of an electrochemical aptamer-based (EAB) sensor specific to vancomycin in phosphate buffered saline (PBS).

FIG. 3 illustrates in graphical form data showing the response of an EAB sensor specific to vancomycin in PBS supplemented with magnesium ions.

FIG. 4 shows square-wave voltammograms and cyclic voltammograms (right panel) obtained during interrogation of an EAB sensor specific to vancomycin.

FIG. 5 illustrates in graphical form the response of an EAB sensor specific to vancomycin to the addition of vancomycin in an artificial interstitial fluid (ISF) without protein content.

FIG. 6 illustrates in graphical form the lack of response of an EAB sensor specific to vancomycin to the addition of vancomycin at some frequencies in an artificial ISF without protein content.

FIG. 7 illustrates in graphical form the response of an EAB sensor specific to vancomycin to the addition of vancomycin in an artificial ISF supplemented with protein.

FIG. 8 illustrates in graphical form the response of an EAB sensor specific to vancomycin to the addition of vancomycin in an artificial ISF supplemented with protein, demonstrating gain correlation with clinical levels of vancomycin.

FIG. 9 illustrates in graphical form the response of an EAB sensor specific to vancomycin to the addition of vancomycin in an artificial ISF supplemented with protein, the EAB sensor being of small dimension.

FIG. 10 illustrates in graphical form the response an EAB sensor specific to vancomycin to the addition of vancomycin in an artificial ISF supplemented with protein, the EAB sensor being of small dimension, demonstrating gain correlation.

FIG. 11 illustrates in graphical form the response of an EAB sensor specific to vancomycin to the addition of vancomycin in human serum.

FIG. 12 illustrates in graphical form the non-response of an EAB sensor specific to vancomycin to the addition of vancomycin in human serum at certain frequencies.

FIG. 13 illustrates in graphical form the response of an EAB sensor specific to vancomycin to the addition of vancomycin in (i) human serum and (ii) artificial ISF.

FIG. 14 illustrates in graphical form the response of an EAB sensor specific to vancomycin of small dimensions to the addition of vancomycin in human serum.

FIG. 15 illustrates in graphical form the response of an EAB sensor specific to vancomycin to the addition of vancomycin in human serum, the EAB sensor being in operable connection with the BlueTooth™ module.

FIG. 16 shows square-wave voltammograms and the cyclic voltammogram (left panel) obtained in human serum with an EAB sensor specific to vancomycin of diameter=170 μm, and in response of to the addition of vancomycin.

FIG. 17 shows in graphical form data comparing the use of two different mathematical approaches for obtaining the dissociation constant of the DNA aptamer (K_{D}) for vancomycin with an EAB sensor specific to vancomycin.

FIG. 18 shows in graphical form the response of an EAB sensor specific to vancomycin (specifically a BASi Au electrode) in artificial ISF supplemented with protein.

FIG. 19 illustrates highly diagrammatically, a therapeutic drug monitoring system configured to monitor drug concentration in the fluid of a subject, and to adjust infusion of the drug by a pump using the concentrations obtained.

FIG. 20 is a photograph that shows a wearable EAB sensor as applied to the skin of the upper arm of a person.

FIG. 21A is a computer-rendered depiction of the surface of the EAB sensor used in the human studies described in Example 6.

FIG. 21B is a diagrammatic cross-section of the EAB sensor of FIG. 21A.

FIG. 22 shows in graphical form the determination of maximum height of a plot of current versus potential.

FIG. 23 shows in graphical form a calibration plot of kinetic differential measurement (KDM) versus log vancomycin concentration.

FIG. 24 shows in graphical form the concentration of vancomycin in the serum and ISF of a single participant (identification code 013) of the human studies described in Example 6. Vancomycin concentration in the ISF was determined by EAB sensor (identification code 811).

FIG. 25 shows in graphical form the concentration of vancomycin in the serum and ISF of a single participant (identification code 001) of the human studies described in Example 6. Vancomycin concentration in the ISF was determined by EAB sensor (identification code 815).

FIG. 26 shows in graphical form the concentration of vancomycin in the serum and ISF of a single participant (identification code 007) of the human studies described in Example 6. Vancomycin concentration in the ISF was determined by EAB sensor (identification code 807).

FIG. 27 shows in graphical form the concentration of vancomycin in the serum and ISF of a single participant (identification code 014) of the human studies described in Example 6. Vancomycin concentration in the ISF was determined by EAB sensor (identification code 810).

Unless otherwise indicated herein, features of the drawings labelled with the same numeral are taken to be the same features, or at least functionally similar features, when used across different drawings.

The drawings are not prepared to any particular scale or dimension and are not presented as being a completely accurate presentation of the various embodiments.

### DETAILED DESCRIPTION OF THE INVENTION AND PREFERRED EMBODIMENTS THEREOF

After considering this description it will be apparent to one skilled in the art how the invention is implemented in various alternative embodiments and alternative applications. However, although various embodiments of the present invention will be described herein, it is understood that these embodiments are presented by way of example only, and not limitation. As such, this description of various alternative embodiments should not be construed to limit the scope or breadth of the present invention. Furthermore, statements of advantages or other aspects apply to specific exemplary embodiments, and not necessarily to all embodiments, or indeed any embodiment covered by the claims.

Throughout the description and the claims of this specification the word “comprise” and variations of the word, such as “comprising” and “comprises” is not intended to exclude other additives, components, integers, or steps.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

The inventors have found that high resolution data having a large number of data points per unit time as obtained from a drug sensor (such as an EAB sensor) is useful for controlling the concentration of drug in the plasma. The present invention may be used to determine an appropriate dosage regimen (e.g., dose and/or dose correction and/or dose interval) for a subject based on observed concentrations of the drug in the subject. In particular, the high resolution data can provide input for a feedback loop including a processor and a drug dosing apparatus (such as an infusion pump) under control of the processor. The drug sensor output is input into the processor and according to program instructions executed by the processor, the pump is controlled so as to adjust a dosage regimen or not controlled so as to leave a current dosage regimen in place.

The high resolution data will provide an indication of when a drug concentration is approaching a threshold, such as an upper or lower concentration of a therapeutic window.

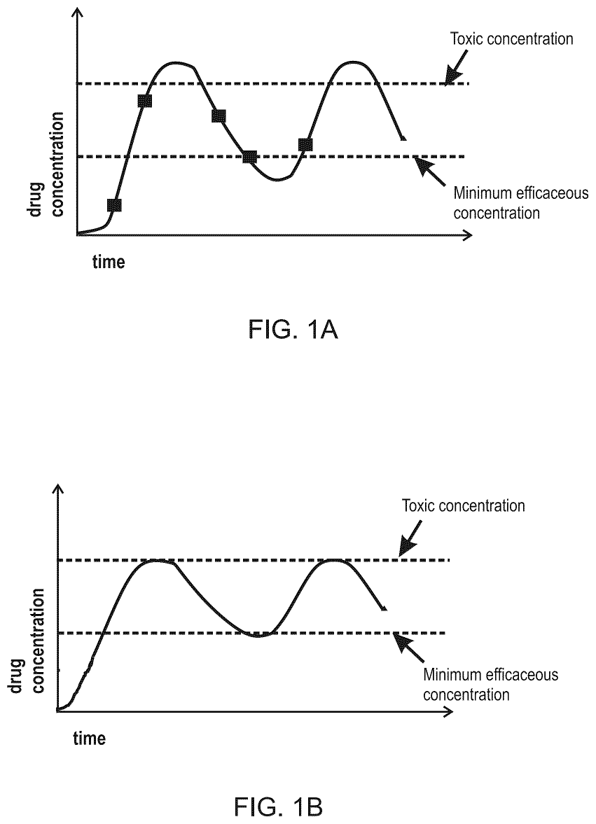

Upon approach of an upper limit, the pump is instantly controlled so as to stop drug administration or to titrate downwardly so as to avoid the drug concentration crossing the upper threshold. Upon approach of a lower limit, the pump is instantly controlled so as to start drug administration or to titrate upwardly so as to avoid the drug concentration crossing the lower threshold. These adjustments to a dosage regimen in response to the high resolution data occur with little or no delay thereby lowering the possibility of a concentration excursion outside the therapeutic window. According to the present invention, the plasma concentration (as indicated by a fluid concentration according to sensor output) is maintained within upper and lower limits, as shown in FIG. 1B.

High resolution sensor data may in some embodiments allow for a drug concentration to be held essentially constant for extended periods of time. The maximums and minimums shown in FIG. 1A and FIG. 1B may be avoided. The continuous infusion of drug in a highly controlled manner so as to achieve a stable level of drug in the circulation is possible where the drug concentration of substantially continuously monitored, and dosage is substantially continuously adjusted instantaneously in light of the sensed concentrations. A steady state is possible given that drug is continually metabolised and/or eliminated from the body, and all that is required is for that drug to be replaced by the infusion pump. In these embodiments, an antibiotic such as vancomycin may be kept at a highly efficacious dosage, set just below the toxic concentration.

The sensor outputs a series of time-based output values, the values being recorded as a data series in electronic memory of the sensor per se, or transmitted to another device having electronic memory (including a proximal smart phone or computer or a remote server, or a cloud server). The output values will typically be a current value that is proportional to the amount of drug present in the fluid under analysis. The raw current value may be used to produce a derivative value (such as drug concentration in ISF) for subsequent use in a pharmacokinetic analysis.

Conversion of drug concentration in the fluid to plasma concentration may be performed. Such conversation may rely on information on how the drug concerned partitions between plasma and fluid. For example, if the fluid is ISF and a drug partitions 2:1 (plasma:ISF), then the ISF concentration is doubled to find the plasma concentration.

Alternatively, data obtained empirically from the subject may be used for the conversion. For example, drug concentration may be obtained from a blood sample and an ISF sample at a point in time. Where the ISF concentration is 3 μg/ml, and the plasma concentration is 3.2 μg/ml a conversion factor of 1.067 is found.

EAB sensor output (whether raw or derived) may be subject to filtering to remove likely outlier values. In addition, or alternatively, the output may be averaged of otherwise smoothed over a period (such as a rolling period) in order to lower the effect of outlier values.

In the sensor output data series, values may be compared to a predetermined maximum value for drug concentration and/or a predetermined minimum concentration. By that comparison, the program instructions are enabled to decide (typically by algorithmic means) whether or not the dosage regime should be adjusted to prevent an excursion. It may only be necessary to slow the pump to avoid breaching the maximum value given that drug is typically continuously metabolised and/or eliminated from the circulation.

Some allowance for a delay in response in plasma drug concentration to a dosage adjustment may be provided. A delay may be expected where the drug is administered by some method other than direct infusion into the circulation, and in which case the drug may need to diffuse into the plasma. Once the sensed concentration is at, say, 95% of the maximum value the drug infusion pump may be stopped or slowed such that the maximum 100% value is achieved (due to existing drug entering the circulation) and at which time the plasma concentration may commence a declining phase. The tight temporal connection between the high resolution data and the immediacy of a response by the pump to adjust dosage will at least in some circumstances mean that little if any allowance for a delay in response in plasma drug concentration to a dosage adjustment will be needed.

As used herein, the term “drug” includes any substance that may be administered to a subject for any prophylactic or therapeutic reason. The drug may be administered by any route and may be detectable in any biological fluid of a subject's body, including but not limited to, ISF, blood, or a mixture thereof.

According to the present invention, EAB sensor output may be sampled and used continuously. As used herein, the word “continuously” in the context of monitoring the level of a drug is intended to include the situation where multiple data points are taken over a monitoring period. A data point may be taken every at intervals measured in nanoseconds, milliseconds, seconds, or minutes. As will be appreciated, it is preferred that short time intervals (such as seconds) are used so as to provide the best opportunity to avoid drug concentration breaching an upper or lower limit. A monitoring period will generally commence around the time the drug is first administered and may extend for minutes or hours.

The EAB sensor may be embodied in many forms, including in the form of a wearable patch or similar having microneedles which extend through the skin surface and into a fluid of the subject in which the drug is detectable.

An EAB sensor may be of the potentiometric, amperometric,or conductometric type. In a potentiometric sensor, a local equilibrium is established at the sensor interface, where either the electrode or membrane potential is measured, and information about a sample is derived from a potential difference between two electrodes. Amperometric sensors rely on a potential being applied between a reference and a working electrode, so as to cause the oxidation or reduction of a redox-active species; with the resultant current being measured. Conductometric sensors rely on the measurement of conductivity at a series of frequencies.

It has been found that EAB sensors are able to reliably and specifically detect a drug in a fluid of a subject. These types of sensors are typically of the amperometric type, with the aptamer (such as DNA, RNA or XNA) being bound to the working electrode. Gold is often used as the probe surface for the working electrode. The aptamer has an associated redox-active species which acts as a reporter. The redox reporter is often methylene blue. Upon target (drug) binding, the aptamer undergoes a conformational change, bringing the redox reporter more proximal to the working electrode surface. This increase in proximity increases electron transfer from the redox reporter to the electrode. The increase in speed of electron transfer contributes to a change in Faradaic current that is detected by a potentiostat.

Aptamers are small (usually from 20 to 60 nucleotides) single-stranded RNA, DNA or XNA oligonucleotides able to bind a target drug with high affinity and specificity. Aptamers may be considered as nucleotide analogues of antibodies, but aptamer production is an in vitro cell-free process that is significantly easier and cheaper than the production of antibodies by cell culture or in vivo methods.

Aptamers are usually selected from combinatorial library having a vast number (up to 10¹⁸) of different oligonucleotides. While RNA aptamers provide a significantly greater structural diversity compared to DNA aptamers, their application is complicated by stability issues in the presence of RNases, high temperature and unfavourable pH.

Selection of an aptamer that is selective for a given drug may be facilitated by a process known as SELEX (systematic evolution of ligands by exponential enrichment). The process may be considered as two alternating stages. In the first stage, the library oligonucleotides are amplified by a polymerase chain reaction (PCR) to the desired concentration. For the selection of RNA aptamers, the single-chained oligoribonucleotides are generated by in vitro transcription of double-stranded DNA with T7 RNA-polymerase. For DNA aptamers, a pool of single-stranded oligodeoxyribonucleotides is generated by strand separation of double-stranded PCR products. In the second stage, the products of amplification are incubated with target drug and oligonucleotides which bind the drug are used in the next SELEX round.

Separation of oligonucleotides with higher affinity for target drug and removal of unbound oligonucleotides are achieved through intense competition for binding sites. The selection pressure rises with every SELEX round. Maximum enrichment of the oligonucleotide pool with aptamers with the strongest affinity for the target molecule is usually achieved after 5 to 15 rounds.

EAB sensors are typically incorporated into a circuit having a reference electrode. The reference electrode is the site of a known chemical reaction that has a known redox potential. For example, a reference electrode based on the silver-silver chloride (Ag|AgCl) redox pair has a fixed and known potential forming the point against which the redox potential of the working electrode is measured. Also typically included in the circuit is a counter electrode which functions as a cathode or an anode to the working electrode. Because the applied voltage bias does not pass through the reference electrode (due to an impedance of the potentiostat), any potential generated is attributed to the working electrode. Current is measured as potential of the interrogating electrode versus the stable potential of the reference electrode. The difference in potential produces the current in the circuit thereby generating an output signal. The signal quantifies target binding depending on electron transfer that is ideally stoichiometrically proportional to target binding.

As discussed above, the EAB sensor may be a microneedle-based patch. When the patch is applied to a subject, the microneedles penetrate the subject's skin contacting a fluid of the subject. The tip of the microneedle functions as the sensor electrode, with the redox reporter tagged aptamer being associated with the tip. This arrangement provides a minimally invasive platform for real-time, continuous in vivo drug detection, which is sufficiently sensitive and selective for monitoring the amount of the drug in the body of a subject over time. EAB sensors are also capable of making single point measurements.

As a microneedle-based patch remain in situ, after being applied to the skin of a subject, the sensor (the tip of the microneedle functionalised with a redox reporter tagged aptamer) remain in continuous contact with the subject's fluid therefore allowing for continuous recording of the drug concentration for the subject. Accordingly, by the present invention, the subject's dosage regime is personalised for to that subject.

While EAB sensors are undoubtedly useful in the context of the present invention, inventors have found that EAB sensors are prone to degradation over time. On each occasion the sensor is interrogated to read a drug concentration a potential is applied to the working electrode, resulting in some loss of aptamer from the working electrode surface. The sensor as a whole loses sensitivity over time, and significantly so over the usual drug monitoring period leading to erroneous output.

While degradation can be limited by restricting the interrogation rate of the working electrode, that approach lessens the number of data points obtained, and therefore the resolution of the data. The present invention may provide means (such as by program instructions) for allowing for the rate of interrogation to be relatively high only when an upper or lower limit in concentration is likely impending. For example, after administration of a drug the sensor may be interrogated every minute. When the drug concentration approaches an upper toxic limit, the rate of interrogation may be increased so as to provide relatively high resolution data. The high resolution data is typically more useful in making a timely decision to adjust the dosage regimen so as to timely avoid any breach of a predetermined limit. Once the drug concentration has started to decline after a peak concentration has been reached, the sensor may be returned to a relatively low interrogation rate such as once per minute. Altering the interrogation rate reduces degradation that would occur if a high interrogation rate were constantly utilised.

The present invention is amenable to computer-implementation given the output of an EAB sensor is an electrical signal that may be electronically stored as a numerical value (e.g., an electrical current value) in volatile memory, and be manipulated and analysed by an associated processor under the instruction of software.

As will be appreciated by the skilled artisan, the present invention may be deployed in part or in whole through one or more processors that execute computer software, program codes, and/or instructions on a processor. The processor may be part of a server, client, network infrastructure, mobile computing platform, stationary computing platform, or other computing platform. A processor may be any kind of computational or processing device capable of executing program instructions, codes, binary instructions and the like. The processor may be or may include a signal processor, digital processor, embedded processor, microprocessor or any variant such as a coprocessor (math co-processor, graphic co-processor, communication co-processor and the like) and the like that may directly or indirectly facilitate execution of program code or program instructions stored thereon.

In addition, the processor may enable execution of multiple programs, threads, and codes.

The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor may include memory that stores methods, codes, instructions and programs as described herein and elsewhere.

Any processor or a mobile device or server may access a storage medium through an interface that may store methods, codes, and instructions as described herein and elsewhere. The storage medium associated with the processor for storing methods, programs, codes, program instructions or other type of instructions capable of being executed by the computing or processing device may include solid state memory and hard disk memory.

A processor may include one or more cores that may enhance speed and performance of a multiprocessor. In some embodiments, the processor may be a dual core processor, quad core processors, other chip-level multiprocessor and the like that combine two or more independent cores (called a die).

The methods and systems described herein may be deployed in part or in whole through one or more hardware components that execute software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, computers, and devices through a wired or a wireless medium, and the like. The methods, programs or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, computers, and devices through a wired or a wireless medium, and the like. The methods, programs or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the invention. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium. The processes, methods, program codes, instructions described herein and elsewhere may be executed by one or more of the network infrastructural elements.

The methods, program codes, calculations, algorithms, and instructions described herein may be implemented on a cellular network having multiple cells. The cellular network may include mobile devices, cell sites, base stations, repeaters, antennas, towers, and the like. The cell network may be a GSM, GPRS, 3G, 4G, 5G, EVDO, mesh, or other network types.

The methods, programs codes, calculations, algorithms and instructions described herein may be implemented on or through mobile devices. The mobile devices may include, cell phones, mobile personal digital assistants, laptops, palmtops, netbooks, pagers, electronic book readers and the like. These devices may include, apart from other components, a storage medium such as a flash memory, buffer, RAM, ROM and one or more computing devices. The computing devices associated with mobile devices may be enabled to execute program codes, methods, and instructions stored thereon.

Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on computer readable media that may include computer components, devices, and recording media that retain digital data used for computing for some interval of time; storage known as random access memory (RAM); mass storage typically for more permanent storage, such as optical discs, forms of magnetic storage like hard disks.

The methods and systems described herein may transform physical and/or or intangible items from one state to another. The methods and systems described herein may also transform data representing physical and/or intangible items from one state to another.

The elements described and depicted herein may imply logical boundaries between the elements. However, according to software or hardware engineering practices, the depicted elements and the functions thereof may be implemented on computers through computer executable media having a processor capable of executing program instructions stored thereon as a monolithic software structure, as standalone software modules, or as modules that employ external routines, code, services, and so forth, or any combination of these, and all such implementations may be within the scope of the present disclosure.

Furthermore, the elements depicted may be implemented on a machine capable of executing program instructions. Thus, while the present descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps thereof, may be realised in hardware, software or any combination of hardware and software suitable for a particular application. The hardware may include a general purpose computer and/or dedicated computing device or specific computing device or particular aspect or component of a specific computing device. The processes may be realised in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realised as a computer executable code capable of being executed on a computer readable medium.

The application software may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, each method described above, and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionalities may be integrated into a dedicated, standalone device or other hardware. In another aspect, the means for performing the steps associated with the processes described above may include any of the hardware and/or software described above. All such permutations and combinations are intended to fall within the scope of the present disclosure.

Any of the methods disclosed herein may be performed by application software executable on any past, present or future operating system of a processor-enabled device such as Windows™, Linux™ Android™, iOS™, and the like. It will be appreciated that any software may be distributed across a number of devices or in a “software as a service” format, or “platform as a service” format whereby participants require only some computer-based means of engaging with the software.

The present invention may be included in a method of treatment, being useful in proposing or even executing a dosage regimen for a drug useful in a particular indication. For example, the method may be for the treatment of bacterial infection with the antibiotic vancomycin as the chosen drug. A method of treatment with vancomycin requires a decision to be made as to rate of infusion, dosage intervals and the like all determined in order to maintain the drug concentration within a therapeutic window. That decision made be made by the clinician after consulting a concentration vs time data or graph generated according to the present invention. Alternatively, the clinician may review a proposed dosage regimen suggested by a software-embodied algorithm or artificial intelligence, with the clinician accepting the proposed dosage regimen and treating the subject accordingly. Alternatively, the clinician may modify the machine-generated proposal based on his/her clinical experience. In some embodiments the method of treatment is completely driven by a system having a drug sensor, algorithmic and/or artificial intelligence capabilities configured to devise a dosage regimen, and means for administering a drug to a subject in a controlled manner according to the regimen.

In some embodiments, machine learning methods are exploited to identify any need to adjust dosage in response to sensor data, or make a decision on how or when a dosage regimen should be adjusted. The machine learning may be human supervised where, for example, in training data a human identifies when a how or when a dosage should be adjusted in consideration of the sensor outputs and any other relevant factor so as to avoid a breach of a concentration limit.

In some embodiments, the machine learns without human assistance to make the relevant dosage decisions. Any of sensor output, the adjustment made to dosage, the effect of the adjust on the concentration, and any observed delay in an effect may be used by a machine learning system to fine tune a later dosage adjustment. By this approach, highly personalised pharmacotherapy is provided that is not based on any assumptions nor any population information.

In terms of generating drug regimens, again a skilled clinician may supervise the machine learning based on training data. In a more sophisticated scenario, machine learning is applied to actual subject data to finely control a drug infusion pump. The machine may learn, for example, that a certain subject exhibits a long delay between drug administration and drug detection in the ISF or blood. Accordingly, the machine may learn to wait for a minimum time period (reflective of the long delay) after drug administration before making any further decision as to whether any more drugs should be administered in order to achieve a minimum effective concentration. By this approach, overshooting the maximum safe concentration of a drug is avoided.

The disclosure from this point in the description and up to the Examples section details various features any of which may be combined with the invention as defined in the Claims section, or in any other part of the present specification. The inventors have also found that high resolution data having a large number of data points per unit time as obtained from a drug sensor (such as an electrochemical aptamer-based sensor) is useful for therapeutic drug monitoring. Such data may be used to determine an appropriate dosage regimen (e.g., dose and/or dose correction and/or dose interval) for a subject based on observed pharmacokinetics of the drug in the subject. In particular, a peak or trough in drug concentration may be accurately identified, and in turn an accurate Cₘₐₓ, Tₘₐₓ, C_{trough} or T_{trough} can be derived and used to determine an appropriate dosage regimen. Furthermore, an impending Cₘₐₓ or Tₘₐₓ may be identified. The inventors have further found that high resolution data obtained from a drug sensor may be used to ascertain the likelihood that an upper concentration or a lower concentration is impending, allowing for an adjustment to an administration regimen so as to prevent any overshoot into an undesirably high or undesirably low concentration.

In an EAB sensor output data series, values may be compared to allow for identification of a peak in concentration. A peak may be identified by, for example, noting the highest output value in a series over a time period for which a peak in drug concentration is expected. For example, where it is known for a drug to have a Cₘₐₓ value in the period of 0.5 to 1.5 hours after administration, then the highest value over that period is taken as the peak. An additional check may be performed which assesses whether the values recorded before the proposed peak are increasing over a significant period, and decreasing for a significant period after the proposed peak. A similar (but inverse) analysis may be used to identify a trough.

An alternative approach would be to identify a trend reversal in values noting where values increase a significant amount over a significant period, with the upward trend reversing with values decreasing a significant amount over a significant period, the highest value over the series representing the peak. A similar (but inverse) analysis may be used to identify a trough.

Once a peak is identified, the sensor interrogation rate may be decreased to limit sensor degradation.

An impending peak may be identified by analysing a series of values over a significant period of time to discern a slowing rate of increase. A significant slowing in the rate of increase may predict that the rate of increase will eventually be zero, at which time the drug concentration would plateau or form a peak. The possibility of a plateau can be effectively ignored where no new drug is being administered and the drug is being transported out of the fluid under analysis by some means, or is subject to metabolism or clearance. Prediction of an impending peak allows for an increase in sensor interrogation rate, which in turn provides a larger number of data points and therefore a more accurate description of the curve about the peak.

An impending trough may be predicted by the administration of a second dosage of drug, and accordingly the interrogation rate of the EAB sensor may be increased. Thus, the interrogation rate may be increased at the time that a second dose is administered in the expectation that an upswing in the drug concentration versus time curve will result.

The may be provided a computer-implemented method for identifying a pharmacokinetic feature for a drug administered to a subject, the method comprising:
contacting a fluid of the subject with an electrochemical aptamer-based sensor capable of detecting the drug,
receiving a series of output values or derivatives thereof of the electrochemical aptamer-based sensor over a period of time, and
using the series of output values or derivatives thereof to identify the pharmacokinetic feature.

Such a method may comprise comparing two or more of the series of output values or derivatives thereof to determine whether the amount of drug in the fluid is increasing, decreasing, or remaining constant.

In one embodiment, the pharmacokinetic feature is a maximum concentration or a time from administration to maximum concentration, and the method comprises identifying a maximum value in the series of outputs or derivatives thereof.

In one embodiment, the maximum concentration is determined by identifying a maximum value in the series of output values or derivatives thereof over a significant time period.

In one embodiment, the pharmacokinetic feature is a minimum concentration, and the method comprises identifying a minimum value in the series of outputs or derivatives thereof in the fluid by reference to the series of outputs or derivatives thereof.

In one embodiment, the minimum concentration is determined by identifying a minimum value in the series of output values or derivatives thereof over a significant time period.

In one embodiment, the series of outputs or derivatives thereof are used to identify a rate of change in the output values or derivatives thereof.

In one embodiment, the rate of change is used to identify a likely impending pharmacokinetic feature.

In one embodiment, the electrochemical aptamer-based sensor is interrogated at a rate, and where a likely impending pharmacokinetic feature is identified, the rate of interrogation is increased.

In one embodiment, the electrochemical aptamer-based sensor is interrogated at a rate, and where a pharmacokinetic feature is identified, the rate of interrogation is decreased.

In one embodiment, the pharmacokinetic feature is identified historically.

There may be provided an apparatus configured to perform for identifying a pharmacokinetic feature for a drug administered to a subject, the apparatus comprising:
an electrochemical aptamer-based sensor configured to contact a fluid of the subject, and
a processor in operable communication with the electrochemical aptamer-based sensor and having access to processor-executable instructions configuring the processor to:
receive a series of output values or derivatives thereof of the electrochemical aptamer-based sensor over a period of time, and
the processor and/or other processor(s) having access to the processor-executable instructions using the series of output values or derivatives thereof to identify the pharmacokinetic feature.

There may be provided a method for determining a clinical action for a subject being treated with a drug, the method comprising identifying a pharmacokinetic feature of the drug in a fluid of the subject by a method disclosed herein, and using the identified pharmacokinetic feature to determine the clinical action.

The clinical action may be selected from the group of continuing administration of the drug, ceasing administration of the drug, altering the dosage of the drug, altering the timing of administration of the drug, altering the rate at which the drug is administered, altering the route of administration of the drug, administering another drug, and ceasing administration of another drug.

There may be provided a method of treating or preventing a condition in a subject, the method comprising administering a drug capable of respectively treating or preventing the condition, identifying a pharmacokinetic feature of the drug in a fluid of the subject by a method disclosed herein, and using the identified pharmacokinetic feature to determine a clinical action.

The clinical action may be selected from the group of continuing administration of the drug, ceasing administration of the drug, altering the dosage of the drug, altering the timing of administration of the drug, altering the rate at which the drug is administered, altering the route of administration of the drug, administering another drug, and ceasing administration of another drug.

The inventors have also found that high resolution data having a large number of data points per unit time as obtained from a drug sensor (such as an electrochemical aptamer-based sensor) is useful for therapeutic drug monitoring. The present invention may be used to determine an appropriate dosage regimen (e.g., dose and/or dose correction and/or dose interval) for a subject based on observed pharmacokinetics of the drug in the subject. In particular, the AUC (i.e. the area under the curve may be identified to provide an accurate picture of exposure of the subject to a drug. Furthermore, an impending peak in the drug concentration curve (Cₘₐₓ) may be identified, and the sensor interrogation rate increased such that the peak is accurately defined by a large number of data points. Before and after the peak, interrogation rates may be relatively low so as to spare the drug sensor unnecessary degradation.

An impending peak may be identified by analysing a series of values or derivatives thereof over a significant period of time to discern a slowing rate of increase. Prediction of an impending peak allows for an increase in sensor interrogation rate, which in turn provides a larger number of data points and therefore a more accurate description of the curve about the peak, and therefore a more accurate AUC.

Where AUC is measured from t=0 to a trough concentration in a multiple drug administration scenario, accurate determination of the trough concentration is provided where a large number of data points define the trough. As for identification of a peak, the rate of interrogation of the sensor may be increased where an impending trough is detected.

The AUC may be calculated (by human or computer means) by any method deemed appropriate by the skilled artisan. An approximation method (such as the trapezoidal method) may be used. For the trapezoidal method, and where a large number of data points are concerned it is most likely that a computer would be used to perform the large number of trapezoidal area calculations required to provide the AUC approximation.

There may be provided a computer-implemented method for determining exposure of a subject to a drug over a period of time, the method comprising:
contacting a fluid of the subject with an electrochemical aptamer-based sensor capable of detecting the drug,
receiving a series of output values or derivatives thereof of the electrochemical aptamer-based sensor over a period of time, and
using the series of output values or derivatives thereof to determine the exposure,
wherein the output values are received at a time interval or an averaged time interval of less than about 4 hours, 3 hours, 2 hours, or 1 hour.

In one embodiment, the exposure is determined by reference to a curve of concentration of the drug over the period of time, the curve generated by reference to the series of output values or derivatives thereof of the electrochemical-aptamer based sensor.

In one embodiment, exposure is determined by determining the area under the curve.

In one embodiment, the period of time commences at or about the time point of administration of the drug, or at or about a time point that the drug reaches a minimal effective concentration, and concludes at a later time point.

In one embodiment, the period of time includes a time point at which a maximum concentration of the drug is present in the fluid.

In one embodiment, the period of time includes a time point at which a minimum effective concentration of the drug is present in the fluid.

In one embodiment, the period of time is the period for which the drug is present in the fluid at or above a minimum effective concentration.

In one embodiment, the period of time concludes at a point in time when a further amount of the drug is administered.

In one embodiment, the administration of the drug, and/or the maximum, and/or the minimum, and/or the minimum effective concentration of the drug, and/or the administration of a further amount of the drug is determined by reference to the series of output values or derivatives thereof of the electrochemical aptamer-based sensor.

In one embodiment, the curve is generated historically.

In one embodiment, the series of outputs or derivatives thereof are used to identify a rate of change in the output values or derivatives thereof.

In one embodiment, the rate of change is used to identify a likely impending maximum or minimum concentration of the drug.

There may be provided an apparatus for determining exposure of a subject to a drug over a period of time, the apparatus comprising:
an electrochemical aptamer-based sensor configured to contact a fluid of the subject, and
a processor in operable communication with the electrochemical aptamer-based sensor and having access to processor-executable instructions configuring the processor to:
receive a series of output values of the electrochemical aptamer-based sensor over a period of time, and
the processor and/or other processor(s) having access to the processor-executable instructions, using the series of output values or derivatives thereof to determine exposure of the subject to a drug over a period of time,
wherein the output values or derivatives thereof are received at a time interval or an averaged time interval of less than about 4 hours, 3 hours, 2 hours, or 1 hour.

There may be provided a method for determining a clinical action for a subject being treated with a drug, the method comprising determining exposure of a subject to a drug over a period of time by the method of any embodiment of the first aspect or using the apparatus of any embodiment of the second aspect, or using the computer-readable medium of any embodiment of the third aspect, and using the determined exposure to determine the clinical action.

In one embodiment, the clinical action is selected from the group of continuing administration of the drug, ceasing administration of the drug, altering the dosage of the drug, altering the timing of administration of the drug, altering the rate at which the drug is administered, altering the route of administration of the drug, administering another or prophylactic therapeutic agent, and ceasing administration of another drug.

There may be provided a method of treating or preventing a condition in a subject, the method comprising administering a drug capable of respectively treating or preventing the condition, determining exposure of a subject to the drug over a period of time by the method of any embodiment of the first aspect, or using the apparatus of any embodiment of the second aspect, or using the computer-readable medium of any embodiment of the third aspect, and using the determined exposure to determine a clinical action.

In one embodiment the clinical action is selected from the group of continuing administration of the drug, ceasing administration of the drug, altering the dosage of the drug, altering the timing of administration of the drug, altering the rate at which the drug is administered, altering the route of administration of the drug, administering another or prophylactic therapeutic agent, and ceasing administration of another drug.

The inventors have also found that high resolution data having a large number of data points per unit time as obtained from a drug sensor (such as an electrochemical aptamer-based sensor) is useful for therapeutic drug monitoring. The present invention may be used to determine an appropriate dosage regimen (e.g., dose and/or dose correction and/or dose interval) for a subject based on predicted pharmacokinetics of the drug in the subject. In particular high resolution drug concentration data provided by a drug sensor (such as an electrochemical aptamer-based sensor) at an early time of the drug concentration curve may be used to predict when and/or to what extent a dosage should be adjusted so as to avoid exceeding an upper level (e.g., a toxic level) or falling below a lower level (e.g., a minimum efficacious level) at a later time.

In the EAB sensor output data series, values may be used to provide kinetic information on the drug in relation to the subject, such information being useful in making a decision (by human or machine) as to whether a dosage regimen should remain or be adjusted and if the latter to what extent the adjustment should be made. For example, the values may be used to determine a rate at which a drug concentration increases or decreases. A rate of change in any rate could be derived to provide an acceleration or deceleration value. Any consistency or inconsistency in kinetic information may also be useful in determining a confidence level in the information or guiding in the need to obtain further data points to give a required level of confidence.

A rate of drug concentration change may be determined from a series of data points commencing at the time drug is administered. The rate in increase in drug can be determined by selecting a number of data points which describe or approximate a straight line and determining the gradient of the line. In some cases, the data points will describe a curve, and in which case multiple straight lines of increasing gradient may be utilised. Alternatively, multiple tangents to the curve may be used to provide a series of increasing gradients.

In some embodiments, the kinetics are not defined in relation to any straight line and instead are described by reference to the formula of a curve which fits the data points.

The kinetic information however obtained will typically be obtained over period at or shortly after the administration of drug. The period may only need be brief (say, several minutes) in order to obtain useful information.

The rate of increase in drug concentration may be used to predict the time at which the drug will approach a predetermined maximum efficacious concentration. If the time is unacceptably distant, dosage may be increased. Otherwise, the dosage may be left unaltered with the rate being used to predict the time that the maximum concentration will be reached. Closer to that time, dosing of the drug may be stopped or decreased to prevent overshoot of serum concentration into a toxic range.

Conversely, after drug concentration has peaked the concentration may head downwardly toward a minimum efficacious concentration. A rate of decrease may be determined shortly after the peak has been reached based on high resolution data from a sensor, and a prediction made as to the time that the minimum concentration will be reached. Closer to the predicted time, drug dosing may be increased so as to prevent drug concentrations moving into a sub-therapeutic range.

By these predictions, a subject's serum concentration for the drug may be maintained within a therapeutic window for an extend period of time.

As used herein, the term “predict” and similar terms are not used in an absolute sense in that any prediction must be confirmed as accurate by later actual data points.

The predictions will typically be made by algorithmic means, the algorithms based on simple maths, empirical data, or theory. A prediction based on the data output by the sensor may be modified by population data or data particular to the subject or some other data.

For example, population data may indicate that subjects of a certain ethnicity tend to have a drug metabolising enzyme that saturates after a short time, and in which case a rate of drug concentration may increase at the time of saturation leading to a faster time in which a toxic concentration of a drug is achieved than would be expected based on the EAB sensor data alone. Thus, the time predicted by the EAB sensor data may be decreased by, say, 10% in an attempt to improve the accuracy of the prediction.

As another example, a subject may have hypotension leading to a low glomerular filtration rate in the kidneys, which in turn leads to a slow clearance of the drug from the circulation. Thus, the drug may accumulate in a more rapid manner that would otherwise be predicted from EAB sensor data with the algorithm taking account of slow clearance when predicting the time at which the drug would cross the threshold concentration and exert toxic effects.

There may be provided a computer-implemented method for predicting a pharmacokinetic feature for a drug administered to a subject, the method comprising:
contacting a fluid of the subject with an electrochemical aptamer-based sensor capable of detecting the drug,
receiving a series of output values of the electrochemical aptamer-based sensor over a period of time, and
using the series of output values or derivatives thereof to predict the pharmacokinetic feature,
wherein the output values or derivatives thereof are received at a time interval or an averaged time interval of less than about 4 hours, 3 hours, 2 hours, or 1 hour.

In one embodiment, the period of time commences at or about the administration of the drug.

In one embodiment, the method comprises comparing two or more of the series of output values or derivatives thereof to determine a kinetic of the drug.

In one embodiment, the kinetic is a rate of increase in the concentration of the drug.

In one embodiment, the kinetic is a change in a rate of increase in the concentration of the drug.

In one embodiment, the kinetic is identified historically.

In one embodiment, the pharmacokinetic feature is a maximum concentration, or a time from administration to maximum concentration, or exposure to the drug.

In one embodiment, the predicting is performed by reference to a subject parameter.

In one embodiment, the subject parameter is selected from the group consisting of weight, age, sex, ethnicity, height, body composition, presence or level of an endogenous agent, presence or level of an exogenous agent, an ability to remove or clear or eliminate or metabolise or inactivate the drug or another agent, renal function, liver function, a disease or condition, a co-morbidity, a genetic factor, and historical data of the subject or a similar subject.

In one embodiment, the predicting is performed at least in part by an algorithm.

In one embodiment, the predicting is performed at least in part by a machine trained to perform the predicting.

In one embodiment, the machine was trained by a machine learning algorithm including a supervised learning algorithm, a semi-supervised learning algorithm, an unsupervised learning algorithm, or a reinforcement learning algorithm.

In one embodiment, the predicting is performed at least in part by artificial intelligence means.

There may be further provided an apparatus for predicting a pharmacokinetic feature for a drug administered to a subject, the apparatus comprising:
an electrochemical aptamer-based sensor configured to contact a fluid of the subject, and
a processor in operable communication with the electrochemical aptamer-based sensor and having access to processor-executable instructions configuring the processor to:
receive a series of output values of the electrochemical aptamer-based sensor over a period of time, and
the processor and/or other processor(s) having access to the processor-executable instructions using the series of output values or derivatives thereof to predict the pharmacokinetic feature,
wherein the output values or derivatives thereof are received at a time interval or an averaged time interval of less than about 4 hours, 3 hours, 2 hours, or 1 hour.

In a third aspect, the present invention provides a computer-readable medium having stored thereon processor-executable instructions as defined in any embodiment of the second aspect.

There may be provided a method for determining a clinical action for a subject being treated with a drug, the method comprising determining using the predicted pharmacokinetic feature to determine the clinical action.

In one embodiment, the clinical action is selected from the group of continuing administration of the drug, ceasing administration of the drug, altering the dosage of the drug, altering the timing of administration of the drug, altering the rate at which the drug is administered, altering the route of administration of the drug, administering another or prophylactic therapeutic agent, and ceasing administration of another drug.

The present invention will now be more fully described by reference to the following non-limiting examples. The invention is described in a non-limiting manner mainly by reference to drug therapy using the antibiotic vancomycin. Vancomycin is a glycopeptide antibiotic effective against gram-positive bacteria and is often used for methicillin-resistant Staphylococcus aureus infection. Whilst effective, vancomycin is nephrotoxic at certain serum concentrations. Vancomycin-associated nephrotoxicity has been found to increase mortality and hospital stay length, especially for subjects having co-existing renal dysfunction. It is therefore of some clinical significance that pharmacokinetic information is provided so as to maintain the drug plasma concentration above the minimal inhibitory concentration but below a concentration where problems of toxicity arise.

### EXAMPLE 1: SENSOR PREPARATION

The experimental protocol included electrochemical cleaning of gold electrodes in 0.5 M NaOH for cyclic voltammetry, as follows:

$${E}_{start} = - 1. V ; {E}_{switch} = - 1.6 V vs Ag \vert AgCl$$

$$v = 1 V {s}^{- 1}$$

200 cycles with Eₛₜₑₚ=2 mV

Electrochemical treatment followed in 0.5 M H₂SO₄:

For platforms that did not require enhancement of sensing area (cyclic voltammetry):

$${E}_{start} = 0 V ; {E}_{switch} = + 1.6 V ; {E}_{fina1} = - 0.2 V vs Ag \vert AgCl$$

$$v = 100 mV {s}^{- 1}$$

Cycle until reproducible voltammograms are obtained:

$${E}_{step} = 1 mV$$

Electrodes were washed 3 times with 1 mL of nuclease free H₂O.

For platforms that required enhancement of sensing area (chronoamperometry):

$${E}_{1} = 0 V ; {E}_{2} = 2 V vs Ag \vert AgCl$$

Pulse length=20 ms
Number of cycles=16,000

Electrodes were washed 3 times with 1 mL of nuclease free H₂O.

Electrode modification was performed according to the following method:
2 μL of 10 mM tris (2-carboxyethyl) phosphine hydrochloride (TCEP) was added to 2 μl of 100 μM DNA-aptamer for vancomycin [5ThioMC6-D/VancomycinDNA/3MeBIN] and kept in the dark for 1 h. Solution was pipetted in/out 5 times.
Adjusted [5ThioMC6-D/VancomycinDNA/3MeBIN] to 500 nM using PBS 1×+2 mM MgCl₂. Solution was pipetted in/out 5 times.
Electrodes were kept immersed in individual solutions for 1 hour in the dark.
Electrodes were washed 3 times with 1 mL of PBS 1×+2 mM MgCl₂.
Incubation followed in in 20 mM 6-mercapto-1-hexanol in PBS 1×+2 mM MgCl₂ overnight at room temperature and in the dark.

### EXAMPLE 2: EXPERIMENTAL STRATEGY FOR EAB SENSOR FOR DETECTION OF VANCOMYCIN

The experimental strategy was to demonstrate sensor viability in macroelectrodes (capable of providing favourable output due to large sensing area) and progress to microelectrodes (having a lower sensing area).

Sensing response analysis was performed initially in a simple matrix (phosphate buffered solution—PBS 1×) and comparatively progressed to more complex matrices that resemble skin interstitial fluid (synthetic interstitial fluid and human serum).

Each combination of electrode/matrix was electrochemically interrogated via square-wave voltammetry to obtain two set of data:
A frequency map, where the signal-on and signal-off frequencies were determined for a particular amplitude.
A titration curve, where the sensor is interrogated at several concentrations of vancomycin by using the optimal signal-on frequency (responsible for the sensing event).

The titration curve also allowed determination of the dissociation constant of the aptamer in the employed matrix.

### Experimental Parameters for Square-Wave Voltammetry

### Frequency and Amplitude Mapping

Eₛₜₐᵣₜ=between −0.5 and −0.4 V
E_{final}=between −0.2 and −0.1 V
E_{ref}=Ag|AgCl

### Additional Parameters:

fix A=10 mV and scan f=5, 10, 15, 20, 25, 30, 35, 40, 45, 50, 55, 60, 65, 70, 80, 90, 100, 150, 200, 250, 300, 350, 400, 450, 500, 600, 700, 800, 900 and 1000 Hz
fix A=25 mV and scan f=5, 10, 15, 20, 25, 30, 35, 40, 45, 50, 55, 60, 65, 70, 80, 90, 100, 150, 200, 250, 300, 350, 400, 450, 500, 600, 700, 800, 900 and 1000 Hz
fix A=50 mV and scan f=5, 10, 15, 20, 25, 30, 35, 40, 45, 50, 55, 60, 65, 70, 80, 90, 100, 150, 200, 250, 300, 350, 400, 450, 500, 600, 700, 800, 900 and 1000 Hz

These processes are performed in the absence and presence of 0.1 mM vancomycin.

### Titration Curve

Eₛₜₐᵣₜ=between −0.5 and −0.4 V
E_{final}=between −0.2 and −0.1 V
E_{ref}=Ag|AgCl

### Additional Parameters:

fix A=optimal amplitude (25 mV for vancomycin-in-vitro)
scan f=optimal signal-on value (between 80 and 100 Hz) and signal-off value (lower than 15 Hz)

### Panel for Dosing Vancomycin In-Vitro (Human Serum)

| Initial Vol. | Concn. (M) | Stock Soln. (M) | Vol. added | Concn. added (M) | Final Vol. |
|---|---|---|---|---|---|
| 1000 microL | 0 1.00E−04 | 2 microL | 2.00E−07 | 1002 microL | |
| 1002 microL | 2.00E−07 | 1.00E−04 | 2 microL | 3.98E−07 | 1004 microL |
| 1004 microL | 3.98E−07 | 1.00E−04 | 2 microL | 5.96E−07 | 1006 microL |
| 1006 microL | 5.96E−07 | 1.00E−04 | 2 microL | 7.94E−07 | 1008 microL |
| 1008 microL | 7.94E−07 | 1.00E−04 | 2 microL | 9.90E−07 | 1010 microL |
| 1010 microL | 9.90E−07 | 1.00E−03 | 1 microL | 1.98E−06 | 1011 microL |
| 1011 microL | 1.98E−06 | 1.00E−03 | 2 microL | 3.95E−06 | 1013 microL |
| 1013 microL | 3.95E−06 | 1.00E−03 | 2 microL | 5.91E−06 | 1015 microL |
| 1015 microL | 5.91E−06 | 1.00E−03 | 2 microL | 7.87E−06 | 1017 microL |
| 1017 microL | 7.87E−06 | 1.00E−03 | 2 microL | 9.81E−06 | 1019 microL |
| 1019 microL | 9.81E−06 | 1.00E−03 | 2 microL | 1.18E−05 | 1021 microL |
| 1021 microL | 1.18E−05 | 1.00E−03 | 2 microL | 1.37E−05 | 1023 microL |
| 1023 microL | 1.37E−05 | 1.00E−03 | 2 microL | 1.56E−05 | 1025 microL |
| 1025 microL | 1.56E−05 | 1.00E−03 | 2 microL | 1.75E−05 | 1027 microL |
| 1027 microL | 1.75E−05 | 1.00E−03 | 2 microL | 1.94E−05 | 1029 microL |
| 1029 microL | 1.94E−05 | 1.00E−03 | 5 microL | 2.42E−05 | 1034 microL |
| 1034 microL | 2.42E−05 | 1.00E−03 | 5 microL | 2.89E−05 | 1039 microL |
| 1039 microL | 2.89E−05 | 1.00E−03 | 5 microL | 3.35E−05 | 1044 microL |
| 1044 microL | 3.35E−05 | 1.00E−03 | 5 microL | 3.81E−05 | 1049 microL |
| 1049 microL | 3.81E−05 | 1.00E−02 | 1 microL | 4.76E−05 | 1050 microL |
| 1050 microL | 4.76E−05 | 1.00E−02 | 2 microL | 6.65E−05 | 1052 microL |
| 1052 microL | 6.65E−05 | 1.00E−02 | 2 microL | 8.54E−05 | 1054 microL |
| 1054 microL | 8.54E−05 | 1.00E−02 | 2 microL | 1.04E−04 | 1056 microL |
| 1056 microL | 1.04E−04 | 1.00E−02 | 10 microL | 1.95E−04 | 1066 microL |
| 1066 microL | 1.95E−04 | 1.00E−02 | 10 microL | 2.88E−04 | 1076 microL |
| 1076 microL | 2.88E−04 | 1.00E−02 | 10 microL | 3.78E−04 | 1086 microL |
| 1086 microL | 3.78E−04 | 1.00E−02 | 10 microL | 4.65E−04 | 1096 microL |

### Panel for Dosing Vancomycin In-Vitro (Artificial ISF)

| Initial Vol. | Concn. (M) | Stock Soln. (M) | Vol. added | Concn. added (M) | Final Vol. |
|---|---|---|---|---|---|
| 10 000 microL | 0 1.00E−03 | 2 microL | 2.00E−07 | 10 002 microL | |
| 10 002 microL | 2.00E−07 | 1.00E−03 | 2 microL | 4.00E−07 | 10 004 microL |
| 10 004 microL | 4.00E−07 | 1.00E−03 | 2 microL | 6.00E−07 | 10 006 microL |
| 10 006 microL | 6.00E−07 | 1.00E−03 | 2 microL | 7.99E−07 | 10 008 microL |
| 10 008 microL | 7.99E−07 | 1.00E−03 | 2 microL | 9.99E−07 | 10 010 microL |
| 10 010 microL | 9.99E−07 | 1.00E−03 | 10 microL | 2.00E−06 | 10 020 microL |
| 10 020 microL | 2.00E−06 | 1.00E−03 | 20 microL | 3.98E−06 | 10 040 microL |
| 10 040 microL | 3.98E−06 | 1.00E−03 | 20 microL | 5.96E−06 | 10 060 microL |
| 10 060 microL | 5.96E−06 | 1.00E−03 | 20 microL | 7.94E−06 | 10 080 microL |
| 10 080 microL | 7.94E−06 | 1.00E−03 | 20 microL | 9.90E−06 | 10 100 microL |
| 10 100 microL | 9.90E−06 | 1.00E−02 | 2 microL | 1.19E−05 | 10 102 microL |
| 10 102 microL | 1.19E−05 | 1.00E−02 | 2 microL | 1.39E−05 | 10 104 microL |
| 10 104 microL | 1.39E−05 | 1.00E−02 | 2 microL | 1.58E−05 | 10 106 microL |
| 10 106 microL | 1.58E−05 | 1.00E−02 | 2 microL | 1.78E−05 | 10 108 microL |
| 10 108 microL | 1.78E−05 | 1.00E−02 | 2 microL | 1.98E−05 | 10 110 microL |
| 10 110 microL | 1.98E−05 | 1.00E−02 | 5 microL | 2.47E−05 | 10 115 microL |
| 10 115 microL | 2.47E−05 | 1.00E−02 | 5 microL | 2.96E−05 | 10 120 microL |
| 10 120 microL | 2.96E−05 | 1.00E−02 | 5 microL | 3.46E−05 | 10 125 microL |
| 10 125 microL | 3.46E−05 | 1.00E−02 | 5 microL | 3.95E−05 | 10 130 microL |
| 10 130 microL | 3.95E−05 | 1.00E−02 | 10 microL | 4.93E−05 | 10 140 microL |
| 10 140 microL | 4.93E−05 | 1.00E−02 | 20 microL | 6.89E−05 | 10 160 microL |
| 10 160 microL | 6.89E−05 | 1.00E−02 | 20 microL | 8.84E−05 | 10 180 microL |
| 10 180 microL | 8.84E−05 | 1.00E−02 | 20 microL | 1.08E−04 | 10 200 microL |
| 10 200 microL | 1.08E−04 | 1.00E−01 | 10 microL | 2.06e−04 | 10 210 microL |

### EXAMPLE 3: RESULTS AND DISCUSSION

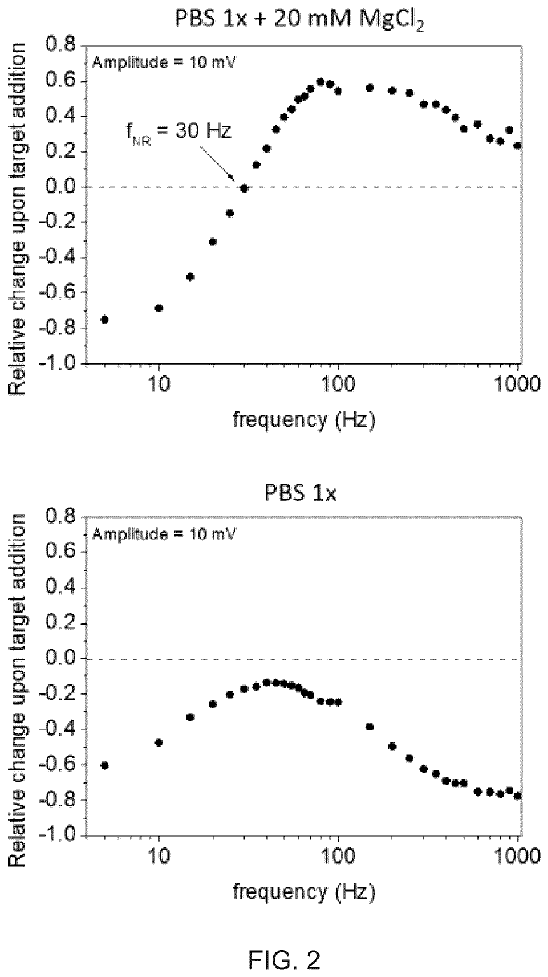

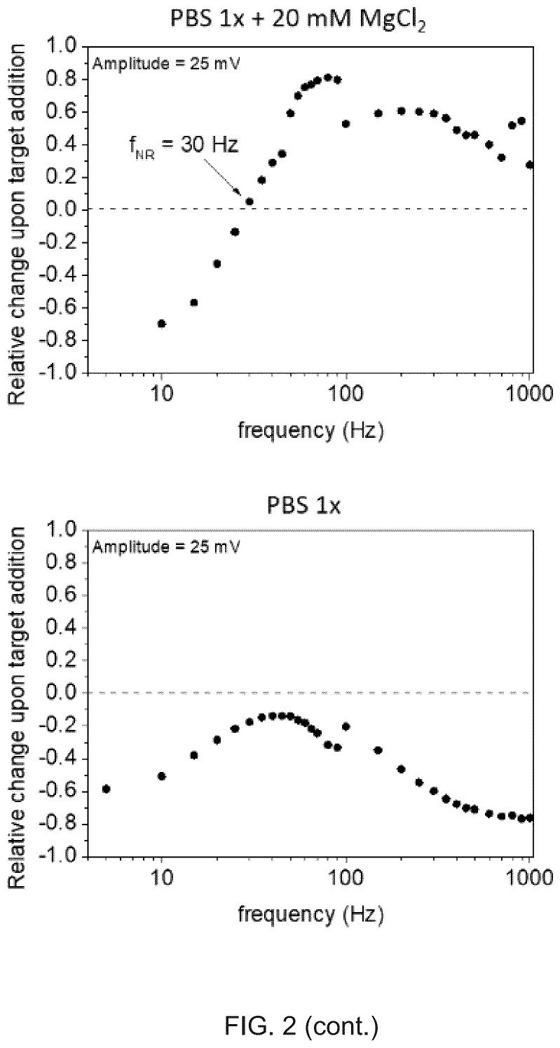

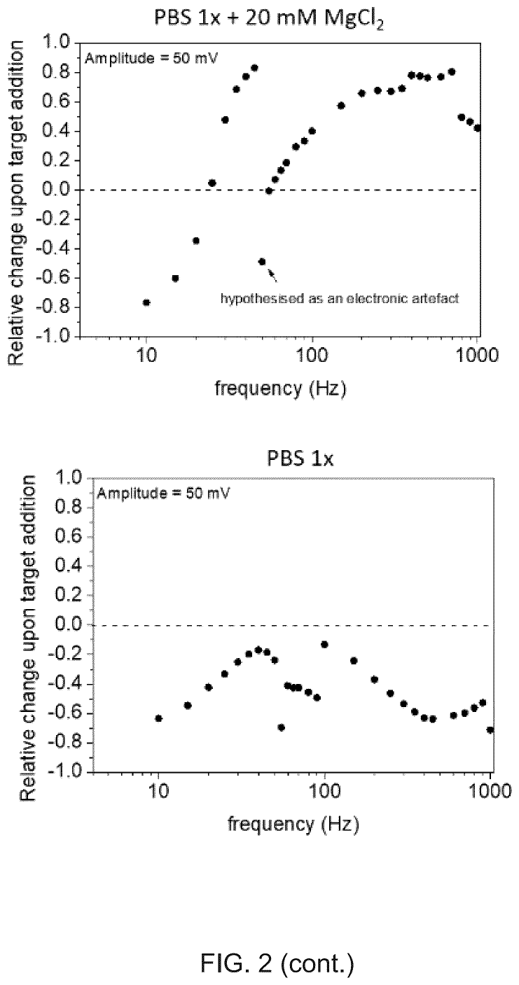

Reference is made to FIG. 2, showing response of a macroelectrode to vancomycin addition in PBS 1×. The data of FIG. 2 shows when gold macroelectrodes (diameter=1 mm) modified with labelled aptamers for vancomycin and antifouling layer were electrochemically interrogated in phosphate buffered solution, signal-on response to analyte dosing was only noted in excess of magnesium cations. Divalent cations are known to influence DNA structures (folding). Discontinuity in data points at 100 Hz are attributed to electronic artefacts due to equipment configuration.

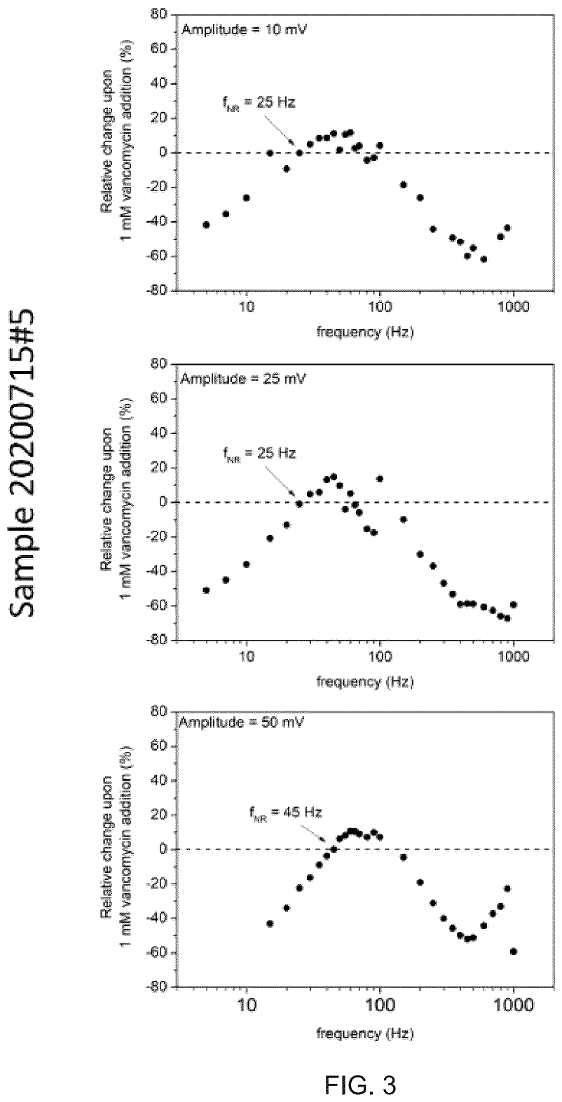

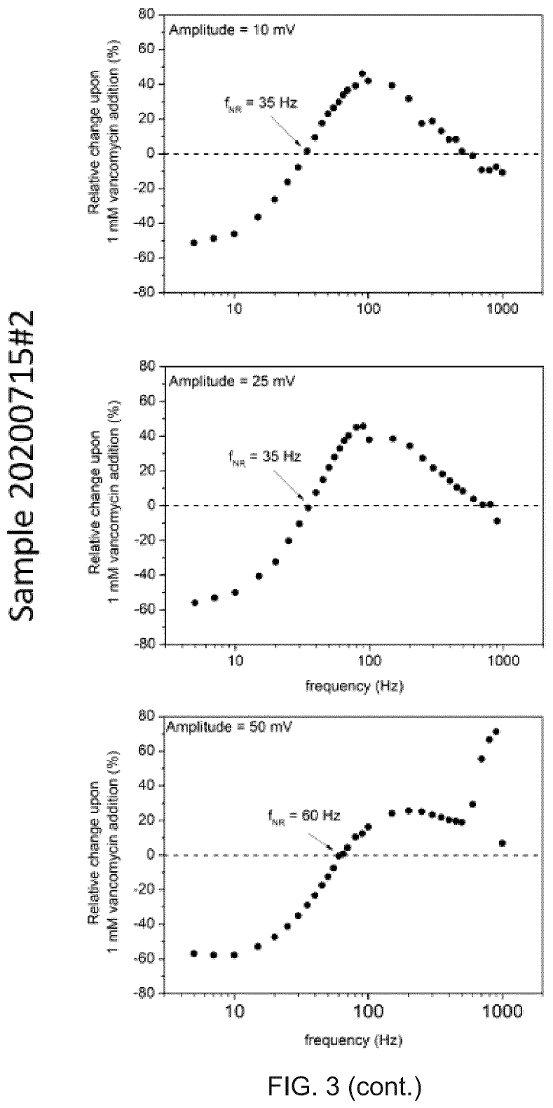

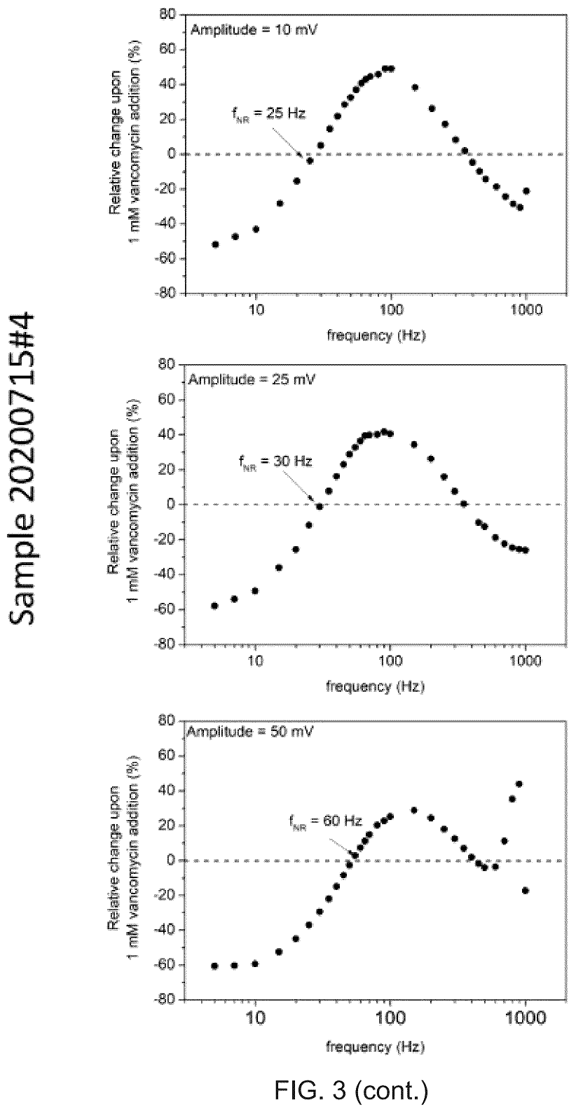

Reference is made to FIG. 3, showing the response of the macroelectrode as from FIG. 2 to vancomycin addition in the presence of 0.7 mM Mg²⁺. The aim of this experiment was to determine whether F_{ON}, f_{NR} and f_{OFF} can be observed in PBS supplemented with 0.7 mM of magnesium cations. Magnesium ions may be found in interstitial fluid at concentrations around 0.7 mM.

The existence of a signal-on, signal-off was noted in the presence of 0.7 mM magnesium cations. The outlier response for Sample 20200115 #5 was attributed to an operator error in sample preparation.

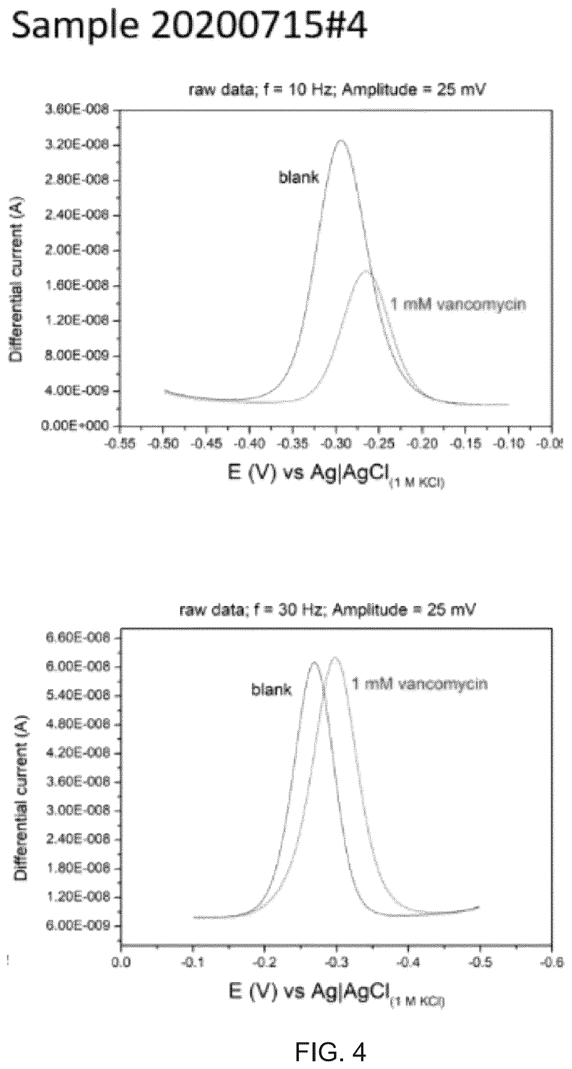

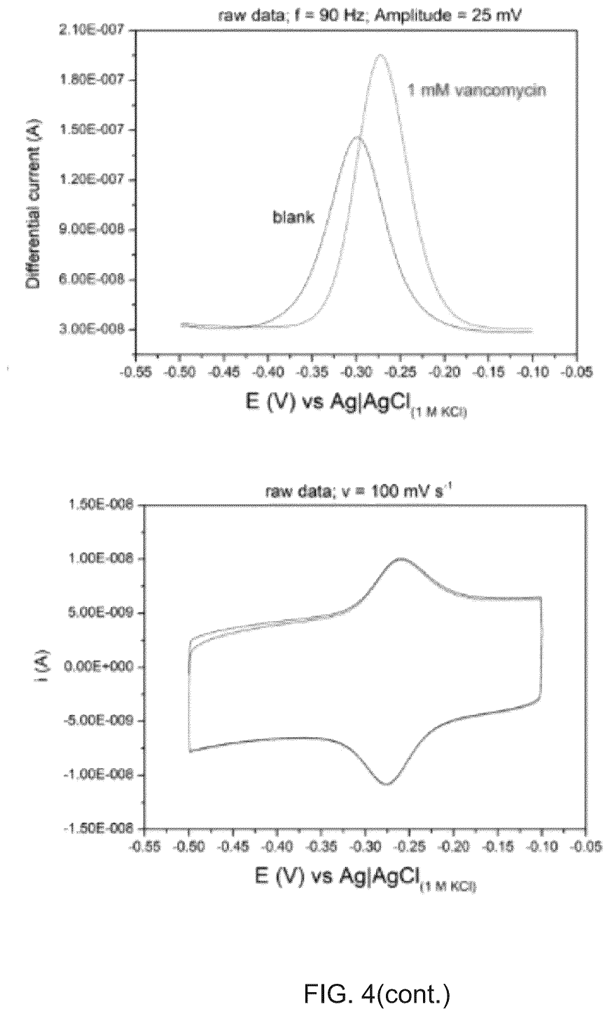

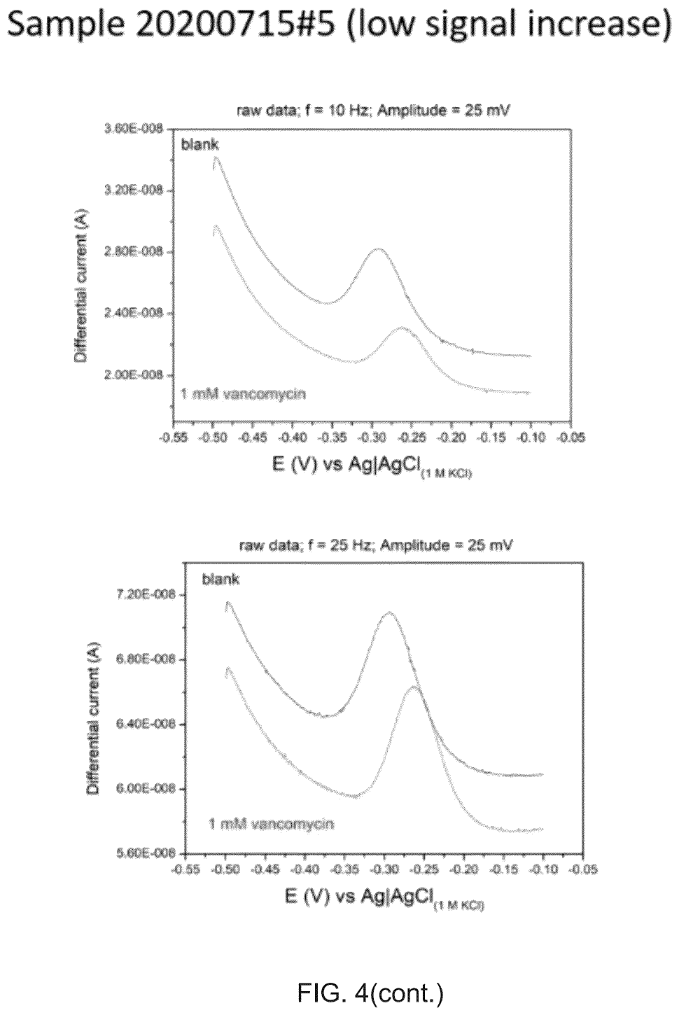

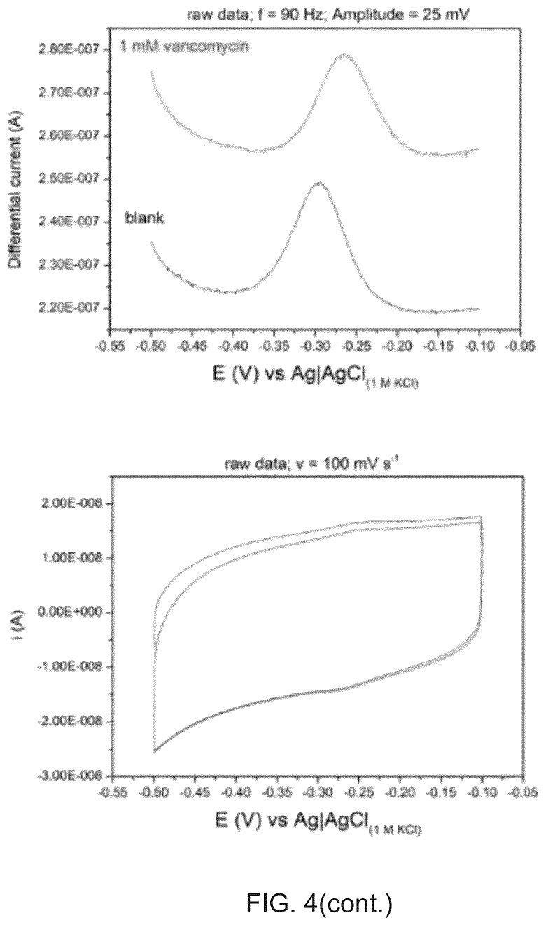

Reference is made to FIG. 4, demonstrating square-wave voltammograms and cyclic voltammograms (right panel) obtained during interrogation of the vancomycin sensing electrodes defined above. The relatively low response of sample 20200715 #5 can be attributed to the lower number (reflected on the magnitude of the peak currents) of labelled aptamers for vancomycin immobilised on the sensor in comparison to sample 20200715 #4.

Reference is made to FIG. 5, showing the response of vancomycin sensing electrodes to the addition of vancomycin in an artificial interstitial fluid without protein content. The existence of a signal-on and signal-off was noted in a solution that emulates the skin interstitial fluid. The composition of this matrix is: 5 mM CaCl₂, 5.5 mM glucose, 10 mM Hepes, 3.5 mM KCl, 0.7 mM MgSO₄, 123 mM NaCl, 1.5 mM NaH₂PO₄ and 7.4 mM saccharose (pH adjusted to 7.35).

Reference is made to FIG. 6, showing the lack of response of vancomycin sensing electrodes to the addition of vancomycin at some frequencies in an artificial interstitial fluid without protein content. The data demonstrates in a synthetic matrix that emulates artificial fluid, there was a correlation between gains obtained from signal-on and signal-off frequencies and the clinical concentration of vancomycin. This allows the preparation of an EAB sensor for vancomycin with minimised interference from drift decay. Data was obtained with an amplitude of 25 mV.

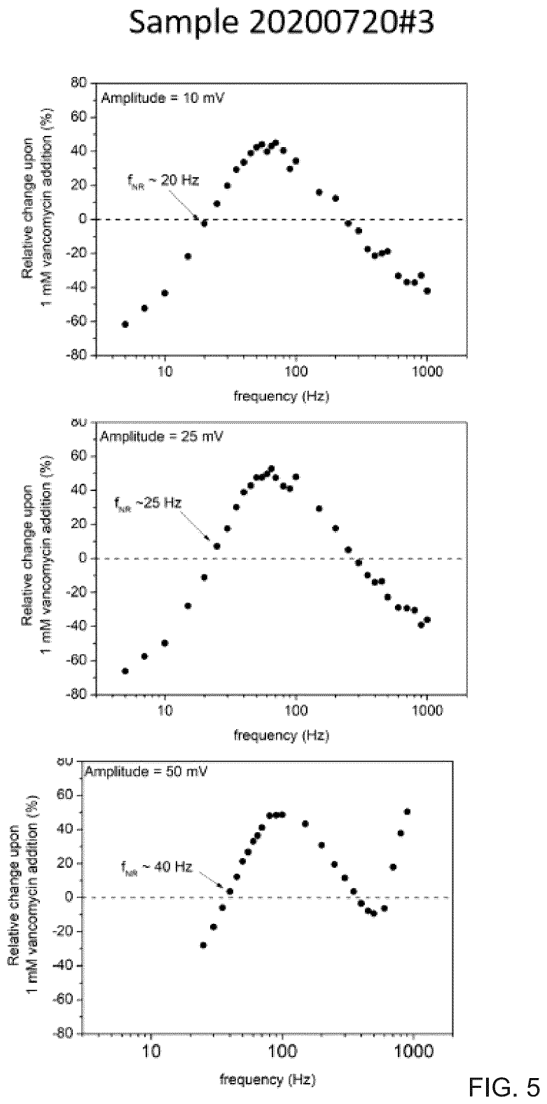

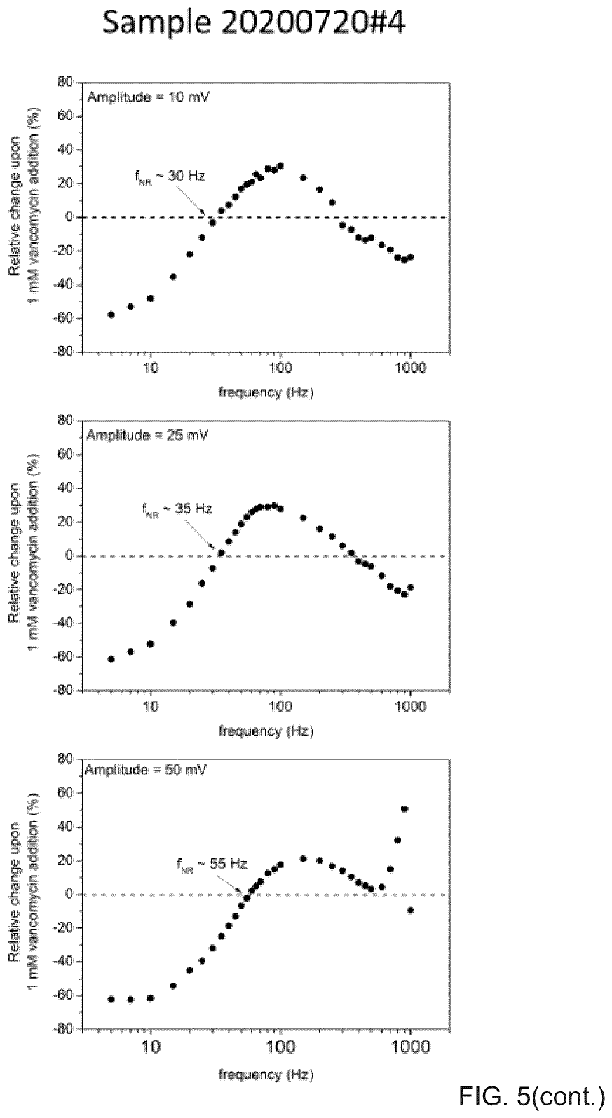

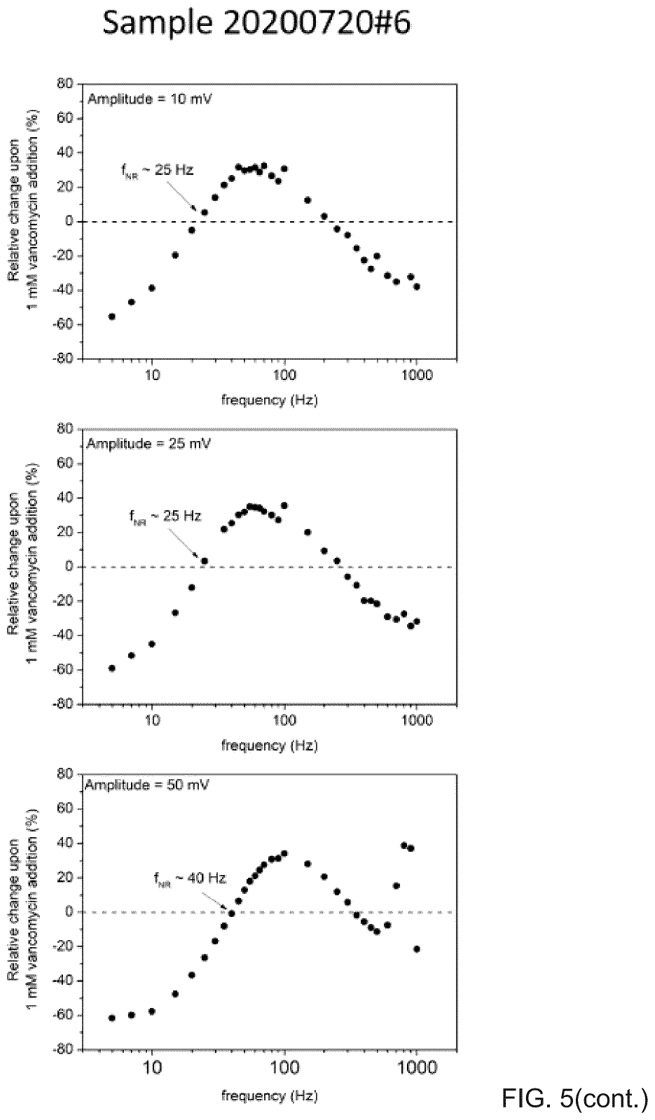

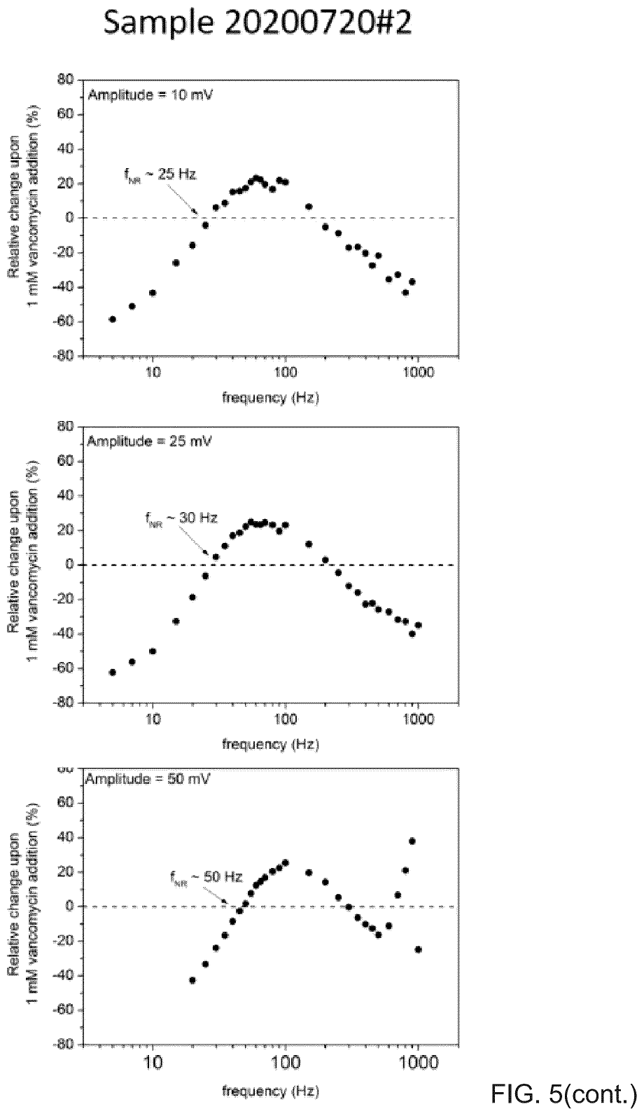

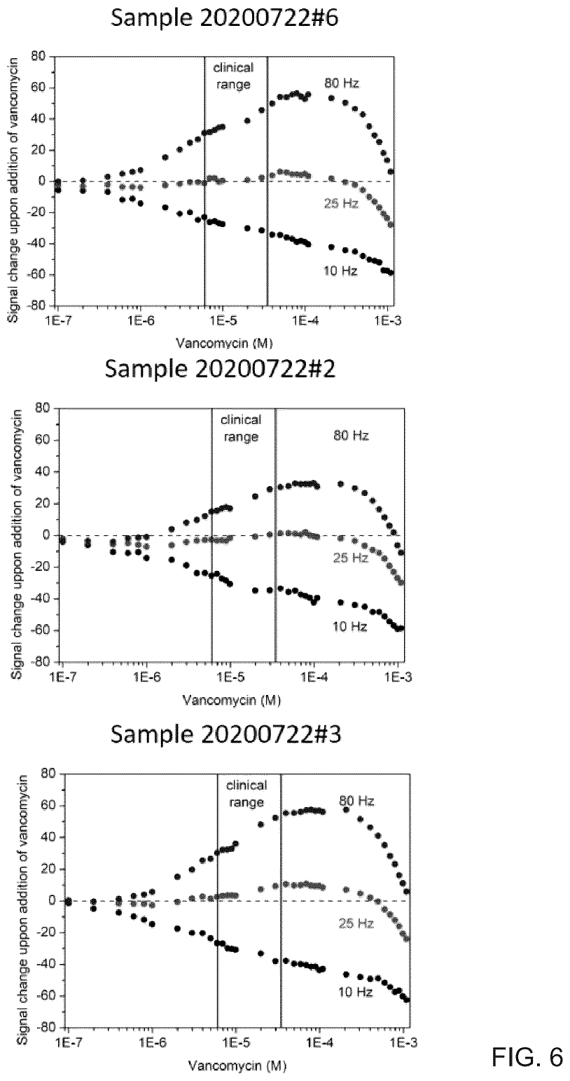

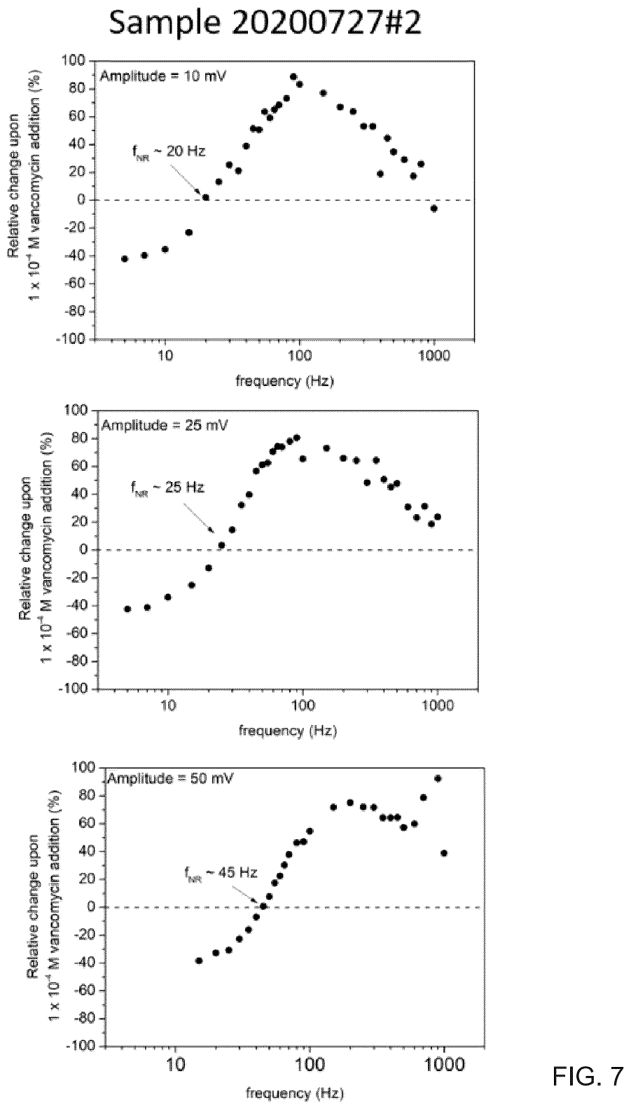

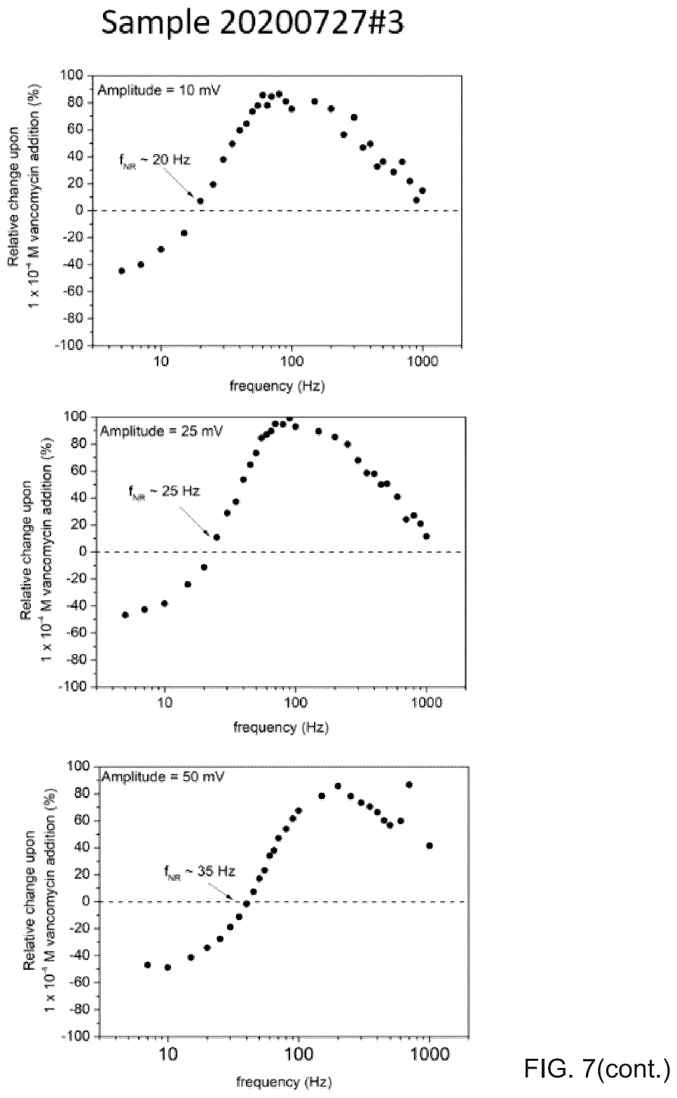

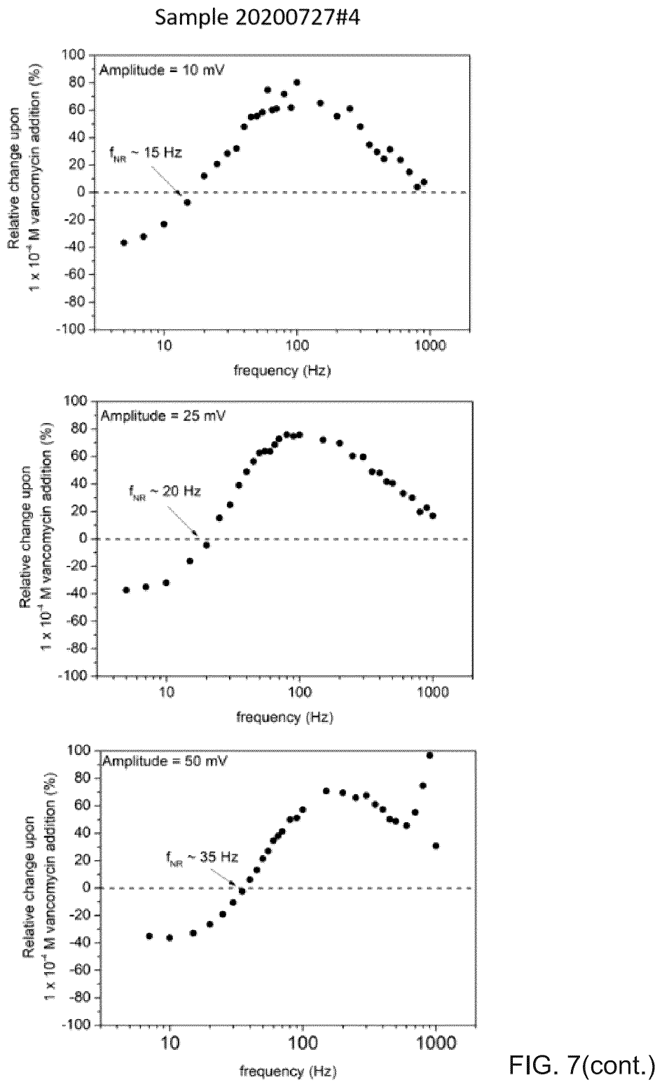

Reference is made to FIG. 7, showing response of vancomycin sensing electrodes to the addition of vancomycin in an artificial interstitial fluid with protein. For a closer emulation of the artificial fluid, the synthetic recipe was altered to include an albumin and globulin protein. The composition of the matrix used was: 107.7 mM NaCl, 3.48 mM KCl, 1.53 mM CaCl₂, 0.69 mM MgSO₄, 26.2 mM NaHCO₃, 1.67 mM NaH₂PO₄, 9.64 mM sodium gluconate, 5.55 mM glucose, 7.6 mM saccharose, 2 mg mL⁻¹ bovine serum albumin and 2 mg mL⁻¹ globulin.

The existence of a signal-on and signal-off was noted in a solution that emulates the skin interstitial fluid in the presence of proteins.

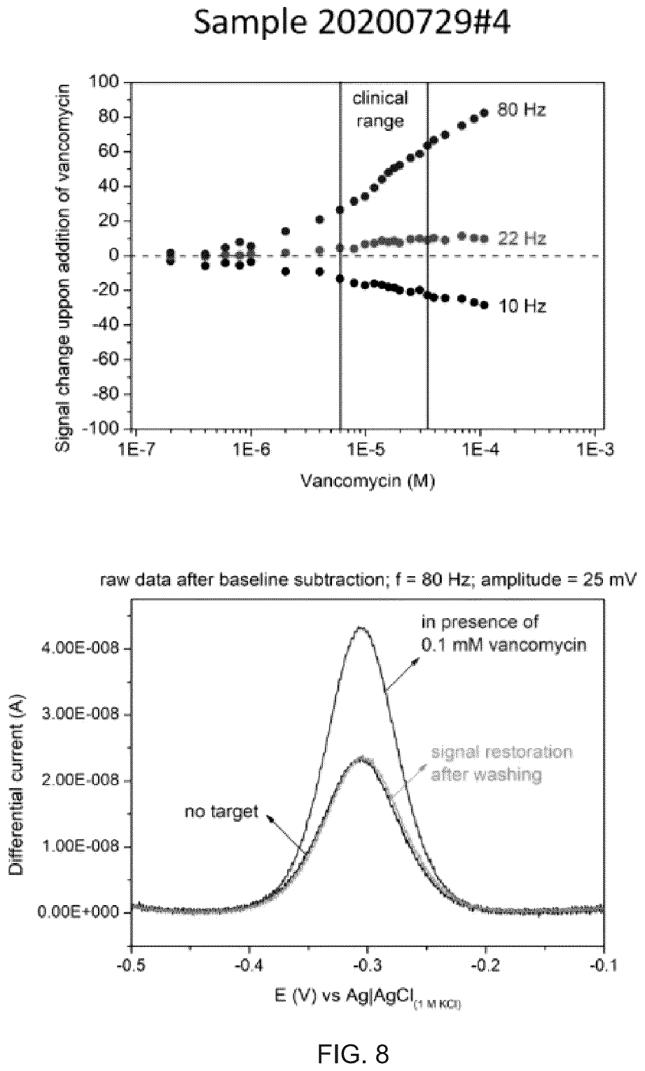

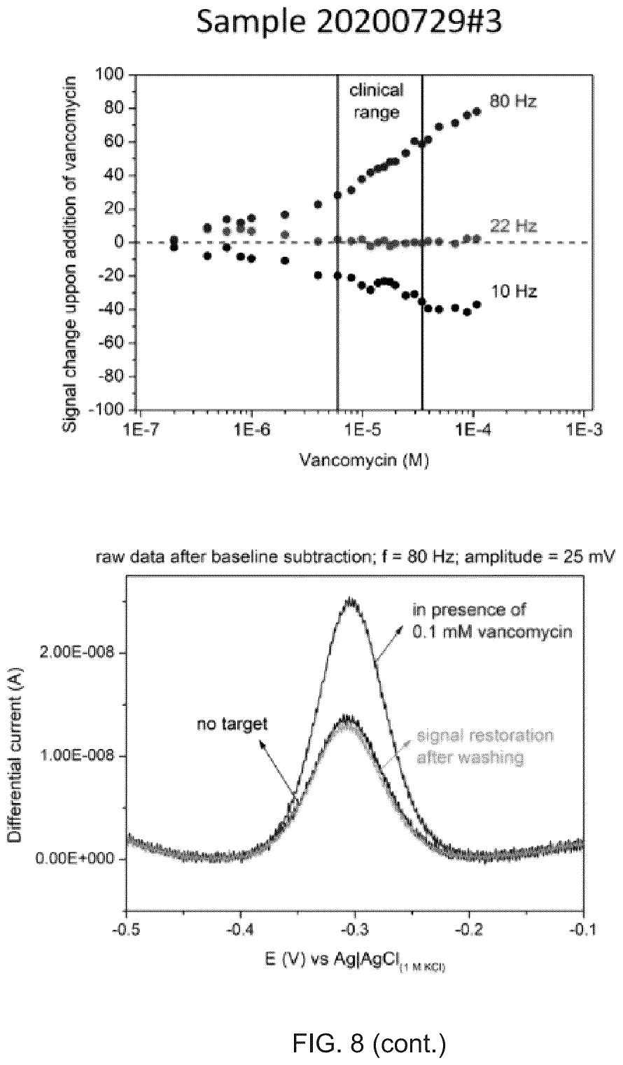

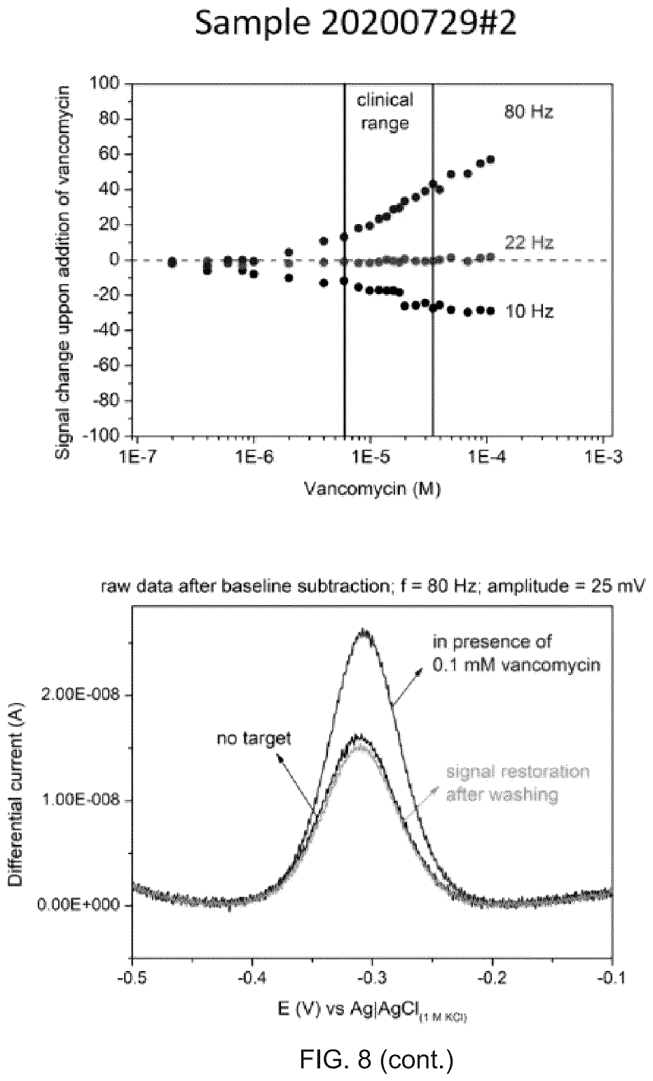

Reference is made to FIG. 8 showing response of a vancomycin sensitive macroelectrode with artificial ISF including proteins. The matrix was the same as for FIG. 6. The data demonstrates that in a synthetic matrix that emulates artificial fluid considering the existence of proteins, there was a correlation between gains obtained from signal-on and signal-off frequencies and the clinical concentration of vancomycin. This allows the preparation of an EAB sensor for vancomycin with minimised interference from drift decay. Data was obtained with an amplitude of 25 mV. The reversible nature of the process is shown by restoring the peak currents to the original value after washing the sensor to remove bounded vancomycin molecules.

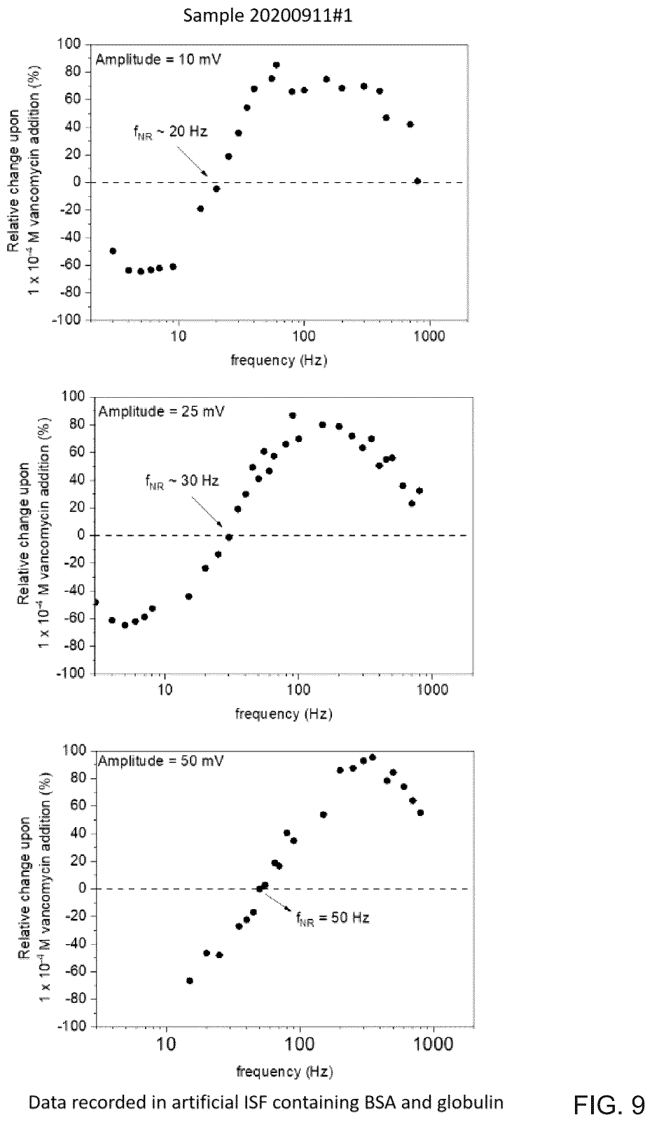

Reference is made to FIG. 9 showing response of a vancomycin sensitive microelectrode with artificial ISF including proteins. The microelectrode employed in this experiment emulates the sensing area of a wearable patch containing 4 microneedles acting as working electrodes (electrodes on which the sensing layer is formed). At such dimensions, the existence of a signal-on and signal-off, was again noted in a solution that emulates the skin interstitial fluid in the presence of proteins.

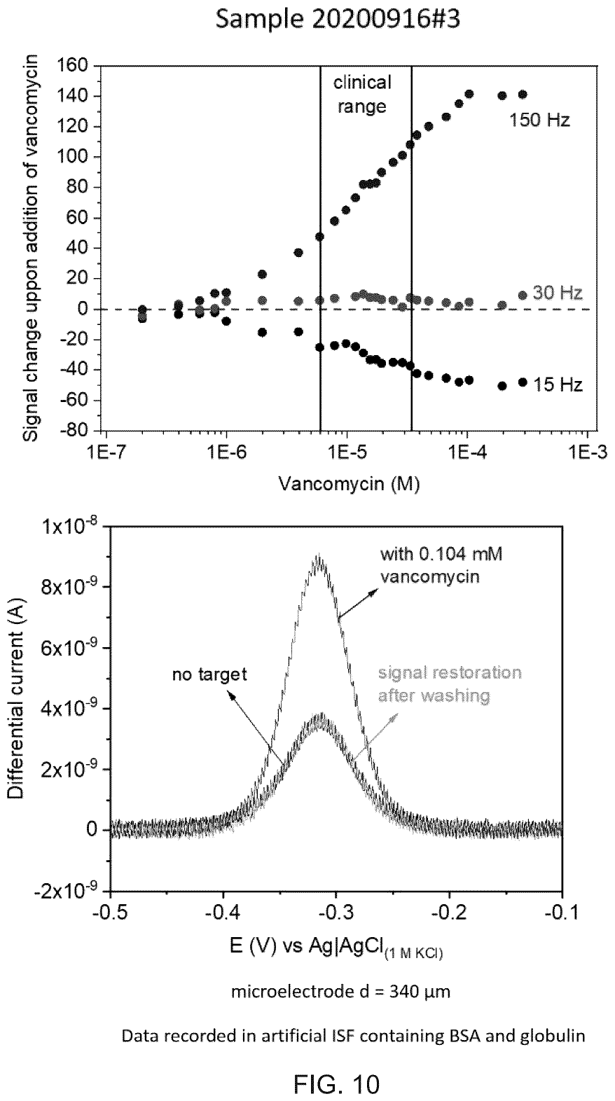

Reference is made to FIG. 10 showing response of a vancomycin sensitive microelectrode (d=340 μm) in a matrix as defined above, being an artificial ISF including proteins. The data shows that in a synthetic matrix that emulates artificial fluid considering the existence of proteins, there was a correlation between gains obtained from signal-on and signal-off frequencies and the clinical concentration of vancomycin. This allows the preparation of an EAB sensor for vancomycin with minimised interference from drift decay. Data was obtained with an amplitude of 25 mV and by using a microelectrode with a sensing area equal to 4 microneedles of a wearable patch. The reversible nature of the process is shown by restoring the peak currents to the original value after washing the sensor to remove bounded vancomycin molecules.

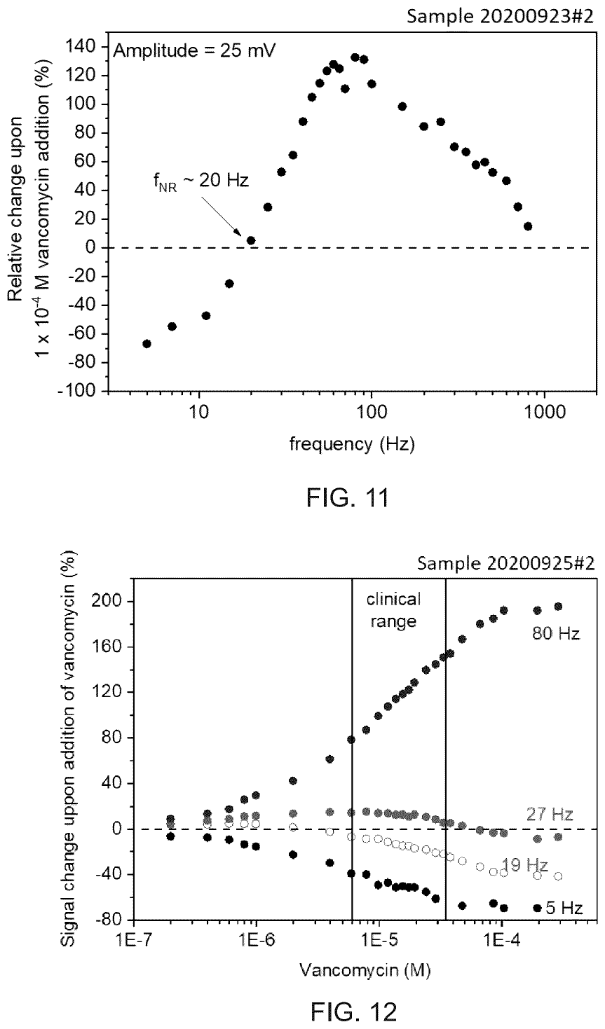

Reference is made to FIG. 11 showing response of a vancomycin sensitive microelectrode (d=340 μm) in human serum. Human serum is a matrix more complex that the synthetic interstitial fluid utilised in the experiments described above. Evaluation of sensing performance in actual human serum provides greater confidence that an electrochemical drug sensor could be useful in a biological fluid as complex as human serum, and in turn actual interstitial fluid. The data shows that with a sensing area matching the use of 4 microneedles as working electrodes, the existence of a signal-on and signal-off was preserved.

Reference is made to FIG. 12 showing non-response at some frequencies to a vancomycin sensitive microelectrode (d=340 μm) in human serum. In human serum, a correlation between gains obtained from signal-on and signal-off frequencies and the clinical concentration of vancomycin was preserved. This allows the preparation of an EAB sensor for vancomycin with minimised interference from drift decay. Data was obtained with an amplitude of 25 mV and by using a microelectrode with a sensing area equal to 4 microneedles of a wearable patch device.

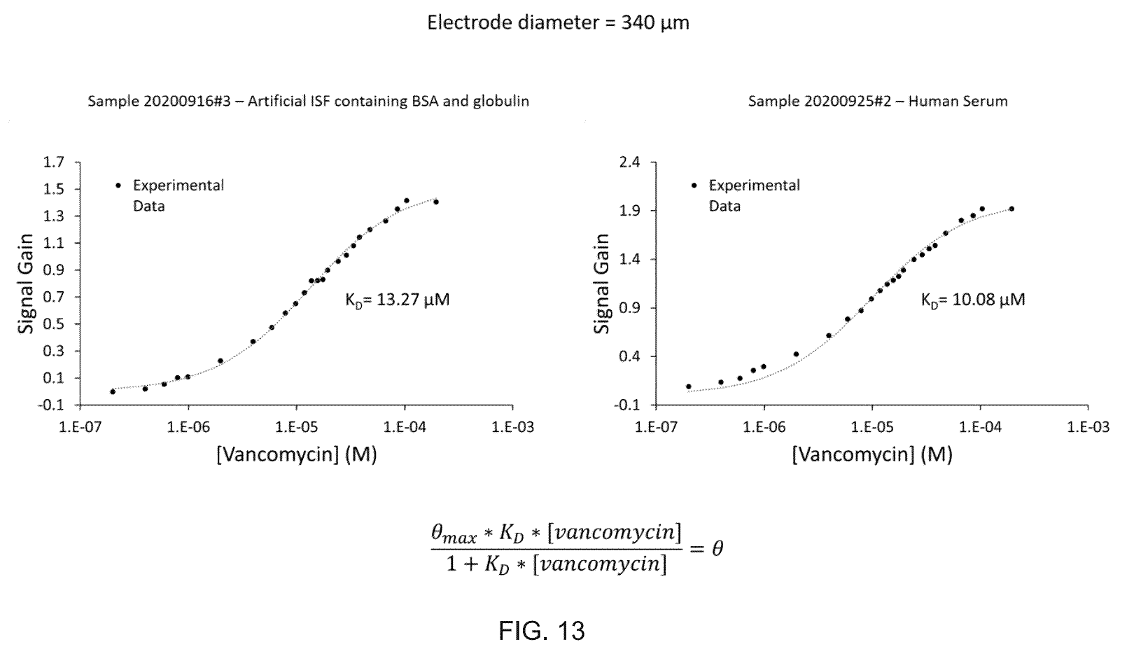

Reference is made to FIG. 13 showing response of a vancomycin sensitive microelectrode (d=340 μm) in (i) human serum and (artificial ISF including BSA, and globulin as described above). The following equation describes an isotherm that represents the response of the sensing layer to the presence of different concentrations of vancomycin.

$$\frac{{θ}_{max} * {K}_{D} * [ vancomycin ]}{1 + {K}_{D} * [ vancomycin ]} = θ$$

The obtaining of the K_{D} (the dissociation constant of the DNA aptamer) was similar in synthetic interstitial fluid containing proteins and in human serum, indicating that the affinity between the aptamer and vancomycin is preserved when the sensor is interrogated in both matrices. Such data suggests that the affinity between aptamer and vancomycin may not be deleteriously affected by employing the sensor in real interstitial fluid.

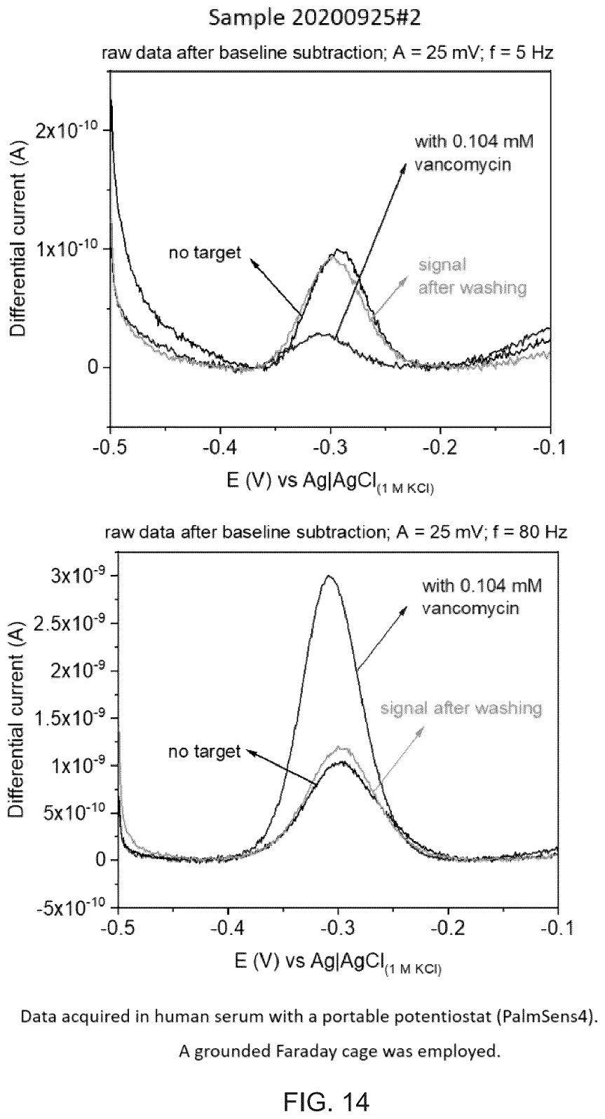

Reference is made to FIG. 14 showing a reversible response of a vancomycin sensitive microelectrode (d=340 μm) in human serum using a portable potentiostat, and employment of a grounded Faraday cage. The reversible nature of the process is shown by restoring the peak currents to the original value after washing the sensor to remove bounded vancomycin molecules.

Reference is made to FIG. 15 showing a response of a vancomycin sensitive microelectrode (d=170 μm) in human serum using a portable potentiostat (PalmSens4™) operating via BlueTooth™, and employment of a grounded Faraday cage. The microelectrode employed in this experiment emulates the sensing area of only one microneedle of a wearable patch device. Even at such small dimensions, the existence of a signal-on, signal-off was noted in human serum.

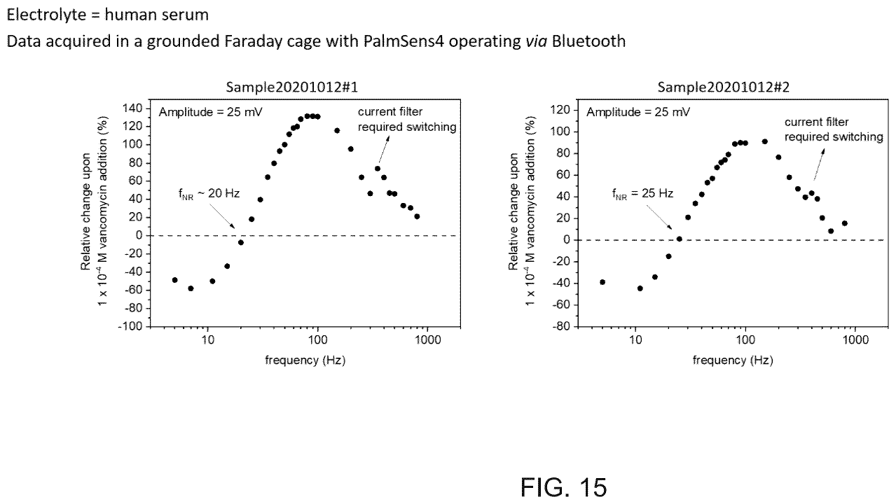

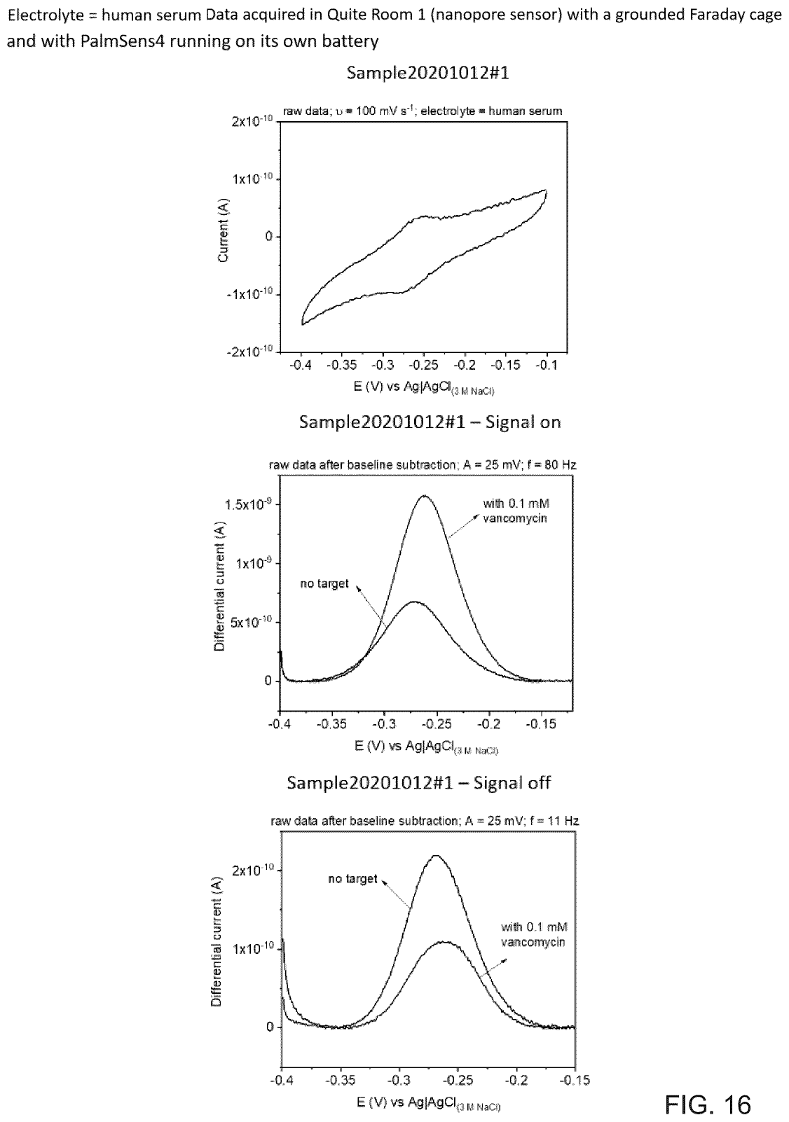

Reference is made to FIG. 16 showing a response of a vancomycin sensitive microelectrode (d=170 μm) in human serum using a portable potentiostat (PalmSens4™) running on its own battery and employment of a grounded Faraday cage. Representative square-wave voltammograms and the cyclic voltammogram (left panel) obtained in human serum with a microelectrode of diameter=170 μm. Data was acquired in Quite Room 1 (nanopore sensor).

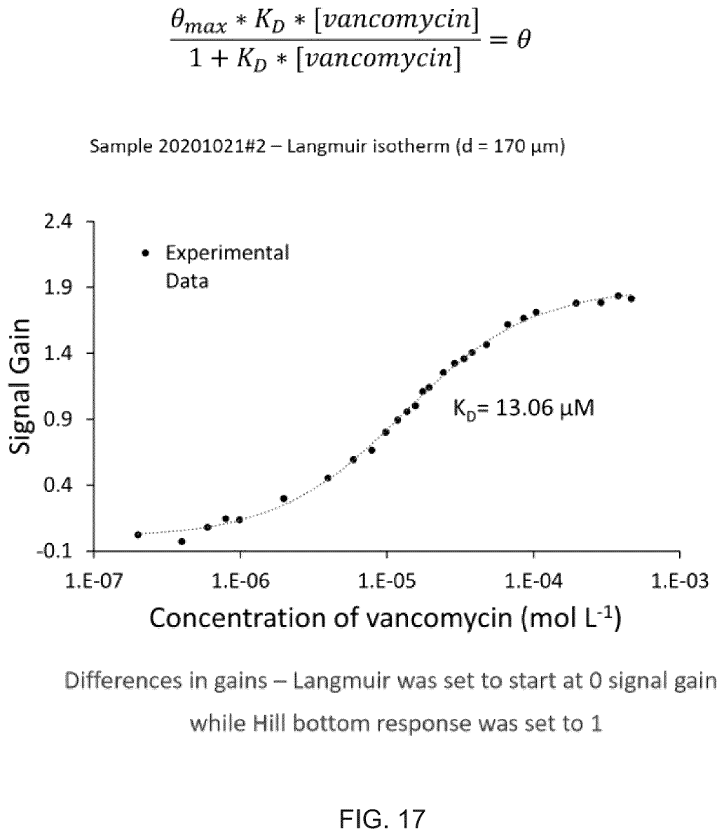

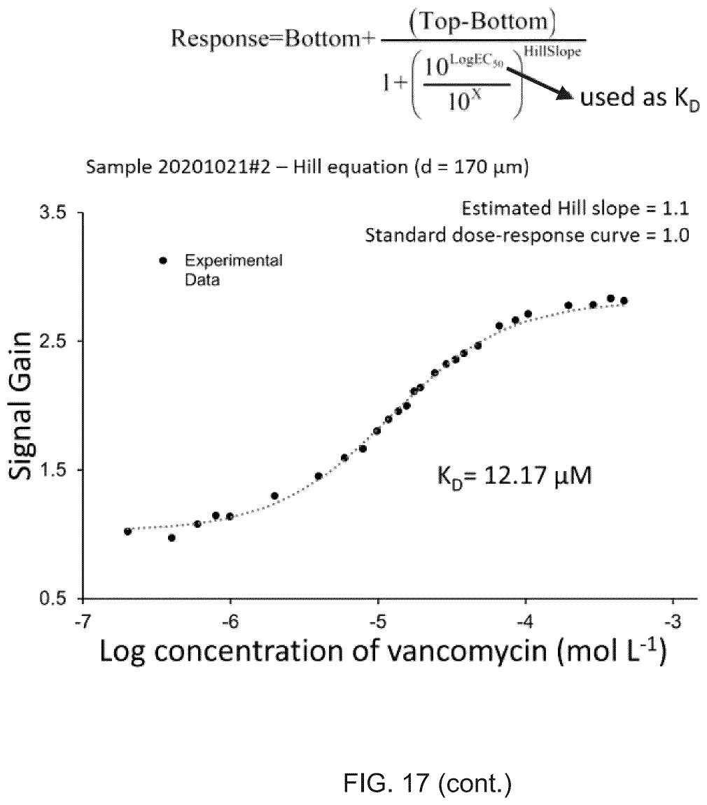

Reference is made to FIG. 17, showing data comparing the use of two different mathematical approaches for obtaining K_{D}, which generated consisting fitting. The K_{D} was preserved at the smaller sensing area, indicating that the affinity between the aptamer and vancomycin was not affected.

### EXAMPLE 4: EXPERIMENTAL PROTOCOL FOR PRODUCING VANCOMYCIN EAB SENSOR USING BASI AU ELECTRODE (d=1.6 mm)

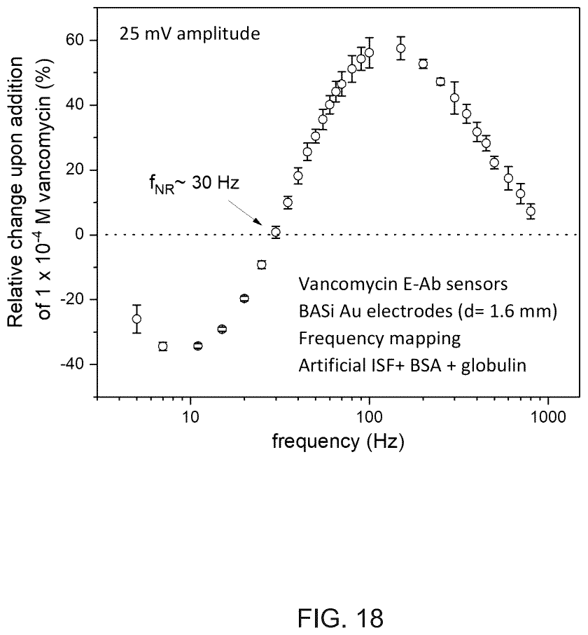

FIG. 18 shows in graphical form the response of vancomycin sensing electrochemical drug sensor (specifically a BASi Au electrode) in artificial ISF supplemented with protein.

In the experimental protocol, gold electrodes were electrochemically cleaned in 0.5 M NaOH, for cyclic voltammetry as follows:

$${E}_{start} = - 1. V ; {E}_{switch} = - 1.6 V vs Ag \vert AgCl$$

$$v = 1 V {s}^{- 1}$$

200 cycles with Eₛₜₑₚ=2 mV

Electrochemical treatment in 0.5 M H₂SO₄:

a) For platforms that do not require enhancement of sensing area (for cyclic voltammetry):

$${E}_{start} = 0 V ; {E}_{switch} = + 1.6 V ; {E}_{fina1} = - 0.2 V vs Ag \vert AgCl$$

$$v = 100 mV {s}^{- 1}$$

15 cycles
Eₛₜₑₚ=1 mV
Electrodes were washed 3 times with 1 mL of nuclease free H₂O.

b) For platforms that requires enhancement of sensing area (for chronoamperometry):

$${E}_{1} = 0 V ; {E}_{2} = 2 V vs Ag \vert AgCl$$

Pulse length=20 ms
Number of cycles=16,000
Electrodes were washed 3 times with 1 mL of nuclease free H₂O.

Electrode modification:
2 μL of 10 mM tris (2-carboxyethyl) phosphine hydrochloride (TCEP) was added to 2 μL of 100 uM DNA-aptamer for vancomycin [5ThioMC6-D/VancomycinDNA/3MeBIN] and kept in the dark for 1 h. Solution is mixed with vortex.
Adjusted [5ThioMC6-D/VancomycinDNA/3MeBIN] to 500 nM using PBS 1×+2 mM MgCl₂. Solution was mixed with vortex.
All electrodes were kept immersed in the exact same solution for 1 hour in the dark.
Electrodes were washed 3 times with 1 mL of PBS 1×+2 mM MgCl₂.
All electrodes were incubated together in 20 mM 6-mercapto-1-hexanol in PBS 1×+2 mM MgCl₂ for a minimum of 2 hours at room temperature and in the dark.

### EXAMPLE 5: SYSTEM FOR THERAPEUTIC DRUG MONITORING AND ADMINISTRATION WITH OPTION OF CONTROLLABLE DRUG INFUSION PUMP

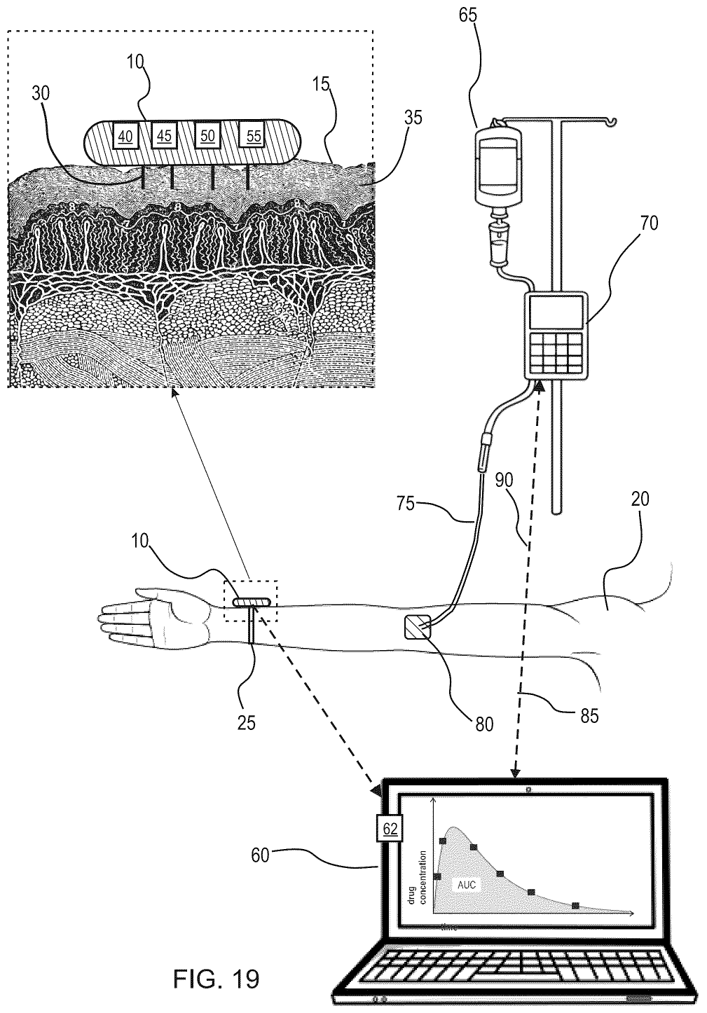

Referring to FIG. 19, there is shown a system for monitoring the levels of a drug with a wearable device and administration of the drug. Particularly, the system comprises a wearable drug EAB sensor device (10) that is retained on the surface (15) of the skin of a subject (20) with an elastic strap (25).

As will be noted from the cross-sectional magnified area inside the dashed square, the wearable device (10) comprises microneedles (one of which is marked (30)). The microneedles breach the stratum corneum which forms the skin surface (15) so as to contact the interstitial fluid of the underlying epidermis (35).

Each of the microneedles (30) is configured as the working electrode of an EAB sensor, and is coated with an aptamer (not drawn) capable of selectively binding to the drug being monitored and administered. Each aptamer molecule has an associated redox reporter configured such that the electrode outputs an electrical signal when drug binds to the aptamer. A power source (40) and circuitry (45) configured to provide electrical power are disposed within the wearable device (10). Also disposed within the wearable device are circuitry (50) configured to receive the signal output by the microneedles (30) and output in digital form to a wireless transmission module (55).

The system comprises a computer (60) having a drug monitoring software (62) executed thereon.

At the commencement of therapy, drug (held in reservoir (65)) is administered via the processor-controlled infusion pump (70), line (75) and cannula (80) to the venous circulation of the subject (20).

From the venous circulation, the drug distributes throughout the body of the subject (20) and a proportion enters the interstitial fluid of the epidermis (35). It is proposed that the level of drug in the interstitial fluid is representative of or proportional to the level of drug presented to target cells of the subject (20).

The drug binds to the aptamer-coated microneedles (30), and the resultant output signal processed by circuitry (50) and then passed to wireless communications module (55). The output signal (which is a drug concentration value) is transmitted wirelessly (85) to the computer (60). The received signal is input into the drug monitoring software under execution by the computer (60).

Given the continuous, real-time output of therapeutic drug concentration by the wearable drug sensor device (10), the therapeutic drug monitoring software (62) is able to determine Cₘₐₓ and Tₘₐₓ for the subject in relation to the drug administered.

The infusion pump (70) is configured to wirelessly transmit (90) therapeutic drug administration data to the therapeutic drug monitoring software (62). For example, the infusion pump (70) may transmit data such as administration start time, administration stop time, and rate of administration to the software (62).

The software (62) may wirelessly transmit (90) instructions to the infusion pump (70) so as to execute a dosage regimen predicted to ensure the therapeutic drug is maintained within a therapeutic window.

### EXAMPLE 6: HIGH RESOLUTION VANCOMYCIN LEVEL DATA IN HUMAN PARTICIPANTS USING EAB SENSOR CONTACTING INTERSTITIAL FLUID

These human studies followed vancomycin levels using an EAB sensor (which may also be referred to as the “device”). The device was a self-contained wearable EAB sensor, constructed, having an on-board power supply, electrodes, and electronics, including a microprocessor and BlueTooth™ communications module.

The EAB sensor comprised four electrodes: two aptamer-coated working electrodes, a counter electrode, and a reference electrode. All electrodes were in the form of a microneedle configured to pierce the skin and contact the participant's ISF when fully inserted into the dermal tissues.

Each of the working electrodes were constructed from a gold plated acupuncture needle, cleaned by a plasma treatment before coating with aptamer in general accordance with the method described at Example 4.

Each working electrode was coated with a vancomycin-sensitive single-stranded DNA-aptamer having a length of 28 nucleobases, obtained from Dr. Milan STOJANOVIC, New York campus research laboratory of The Trustees of Columbia University in the City of New York of 80 Claremont Avenue, 4th Floor, New York, NY 10027, United States. The DNA-aptamer was previously demonstrated to specifically interact with vancomycin.

Each entire device was disinfected by immersion for 10 minutes in Cidex™ (Johnson & Johnson) at room temperature. Devices were then rinsed in sterile phosphate buffered saline for 30 seconds.

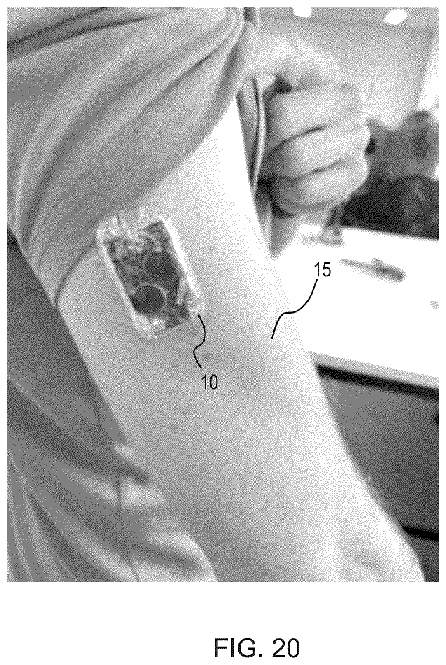

The device comprised an adhesive disposed on a surface surrounding the electrodes, maintaining the device on the skin and therefore keeping the electrodes in contact with the interstitial fluid (see FIG. 20).

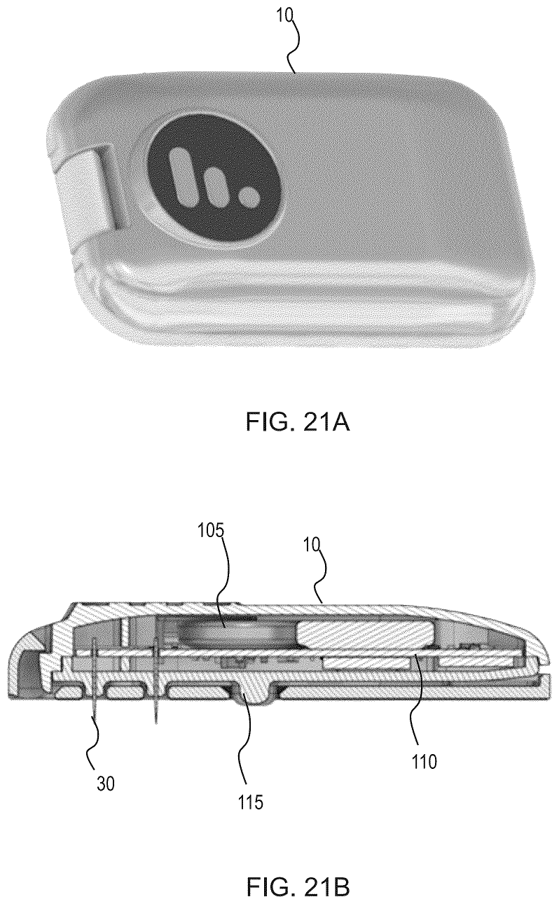

Reference is made to FIG. 21A and FIG. 21B each showing a computer-rendered representation of the device (10) used in these studies, indicating one of the microneedles (30), the power supply, being a battery (105), a printed circuit board (110) having various electronic components such as the communications module and microprocessor mounted thereon, and a thermistor (115) functioning to contact the skin surface of the participant and provide an estimation of the temperature of the underlying ISF.

These human studies were performed at Monash Health, Monash Medical Centre, Clayton, Victoria 3168, Australia, under protocol reference number 2021/ETH80521 and protocol trial identified and registry 80521. Institutional Ethics Committee approval was obtained before commencement of the study.

Selection criteria used were as follows. Age 18-60 years. Individuals without clinically significant medical abnormalities contraindicating participation as determined by Study Investigators, including, but not limited to: (a) physical examination without any clinically relevant findings, (b) systolic blood pressure in the range of 90 to 140 mmHg (inclusive) and diastolic blood pressure in the range of 50 to 90 mmHg (inclusive) after 5 minutes of rest in a supine position, (c) pulse rate in the range of 60 to 100 bpm (inclusive) after 5 minutes of rest in a supine position. A 40-60 bpm (inclusive) may be considered acceptable for participants without clinically significant findings at the discretion of the Principal Investigator, (d) body temperature (tympanic), between 35.5° C. and 37.5° C. (inclusive), (e) no clinically significant findings in serum biochemistry, haematology tests, or urinalysis contraindicating participation as determined by Study Investigators.

Female participants of childbearing potential were required, from the period of signing the consent form until at least 28 days after the removal of the device: (a) to have a negative pregnancy test at screening and study visits, (b) not to be planning to become pregnant, (c) not to be breastfeeding, (d) not to donate ova. If engaging in sexual intercourse, they were required to use effective contraception during the studies and strongly recommended to use effective contraception for at least 28 days after the removal of the device.

Participation was not influenced by vaccination status. However, participants who had been vaccinated within one week of the study visit, were not recruited.

Exclusion criteria were as follows. Poor venous access for venepuncture. Participants who were currently receiving or have received any investigational drug/device within the last 30 days. History of allergic reactions to vancomycin, metals, plastics and adhesives which, in the opinion of the Study Investigators, would increase the risk of having allergic reactions associated with skin allergies, or vancomycin administration. Active illnesses. Consumption of prescription medications except oral contraceptive pills. Use of illicit drugs or alcohol consumption, which, in the opinion of the Study Investigators, may interfere with the completion of the studies.

A vancomycin-sensitive EAB sensor was placed onto the participant's upper arm (see FIG. 20), opposite the arm where vancomycin was being infused. The time of device application was recorded. The device was applied 30 minutes (+15 minutes) prior to administering the vancomycin infusion and the device was removed up to 10 hours post-cessation of the infusion.

Blood samples for relevant pathology tests (FBC, UEC, and LFT) were collected (10 to 30 minutes prior to application of the device and 10 to 30 minutes post-removal of the device). If the participant consented to providing blood samples for future research, these were collected prior to the placement of the device, or once the device has been removed, at the time most convenient for the Clinical Team.

30 minutes (+15 minutes) post-application of the device, the participant received a single dose of vancomycin as an intravenous infusion (1 gram over 1 hour 40 minutes). The time of administration and completion of infusion was recorded.

Blood samples for the measurement of vancomycin concentrations were collected prior to the administration of vancomycin infusion, 30 minutes (±5 minutes) and 1 hour (±10minutes) during the infusion, at the end of the infusion (±15 minutes) and then at 40 minutes (±15 minutes), 1.5 hours (±15 minutes), 2 hours (±15 minutes), 3 hours (±15 minutes), 4 hours (±15 minutes), 6 hours (±15 minutes), 8 hours (±15 minutes) and 10 hours (±15 minutes) after the infusion has been completed.

Participants were required to complete a pain scale survey 5-10 minutes post-application and 5-10 minutes after removal of the device.

Participants were required to conduct a physical challenge post-application of the device.

5-10 minutes prior to removal of the device, a mobility survey was completed.

Digitally captured images/recordings of the skin surface at the device application site, before and after application and removal of the device, were taken to assess any skin irritation.

Participants were monitored throughout the study duration for any adverse events. Given the duration of the study visit, participants were required to stay overnight. In the absence of any adverse events, participants were discharged the following morning after being observed for at least 15 minutes post removal of the device.

Application of the device for any reason and at any stage of the studies was as follows. Device application location was cleaned thoroughly with alcohol wipe (provided) as if the site were to be the site of an injection. The area was allowed to dry for 10 to 15 seconds before applying the device. An adhesive liner was removed from the bottom of the device being careful not to remove the safety tab. Prior to applying the device, the safety tab was still in the device. The device was applied to the cleaned site. Firm pressure was applied to the top of the device for 5 to 10 seconds. The device was applied with the safety tab pointing up. The safety tab was removed, and the top of the device was pressed such that the microneedles penetrated the skin. An audible click was heard as the device is pressed down, indicating that the microneedles were fully extended and locked in place.

Once applied to the participant, the DNA-based sensor electrodes were interrogated, and output treated as follows.

Square Wave Voltammetry (SWV) was used to interrogate the DNA-based sensor electrodes. Multiple steps were performed to convert the raw voltammograms obtained from the DNA-based sensor electrodes into a vancomycin concentration. The steps detailed below convert the raw SWV voltammograms into a signal that is indicative of the analyte concentration.
1. Smooth the raw SWV voltammogram current versus voltage data to aid identification of the current peak and its magnitude.
2. Apply a peak finding algorithm to the smoothed voltammogram to identify the position of the peak and to subtract a baseline current to determine a peak current magnitude.
3. Use the determined peak currents magnitudes obtained at two different SWV interrogation frequencies (in this case 50 Hz and 300 Hz), when no vancomycin is present and when it is present to calculate an analyte concentration responsive signal(S).
4. Smooth the S values over time, prior to applying a calibration function.

Steps 1 to 3 were used to analyse the calibration data as detailed below at (a) and (b), and steps 1 to 4 above in relation to the clinical data.
(a) Calibration data was produced from in vitro testing, where vancomycin is spiked into bovine plasma and tested using electrodes from the same production batch as those used in the clinical experiments (but not the electrode actually used in the clinical experiments). The purpose of this testing was to produce S versus known vancomycin concentration data that can be used to produce a calibration function to convert the S values into the corresponding measured vancomycin concentration values ([V]).
(b) Clinical experiment data, where the calibration function determined in (a) is applied to the S values produced by the in vivo electrodes in the clinical experiment, to convert them to measured vancomycin concentrations. This process produces [V] over time data.

The last step for the clinical data is to calculate the average [V] value of the two sensing electrodes (e.g., working electrodes) in the same device at each time point to reduce random variation to give the final vancomycin concentration estimate.

Further detail in the process for generating vancomycin concentration as outline above will now be provided.

### Voltammogram Smoothing

The first part of the peak-finding process is to smooth the measured current data. This reduces noise and makes identification of peaks easier.

Smoothing was performed using a Savitzky-Golay filter. This filter moves along the array and fits a polynomial curve to a sliding window. Testing has shown that this filter performs well at eliminating noise but also retains peaks and troughs better than a rolling average approach. The raw data appears to tolerate aggressive filtering well thereby simplifying downstream peak-finding.

### Baselining and Peak Measurement

The next step was to simply interpolate a baseline between the left and right troughs and identify the peak which is the maximum difference between the curve and the baseline when measuring vertically (and not perpendicular to the baseline).

The baselining approach uses the following process:
(i) Start at the left and right extremes of the curve.
(ii) Attempt to draw a straight line between the two points from the left to the right.
(iii) If at any point along this baseline, the actual curve is below the baseline then stop and move the left point one in along the curve.
(iv) Attempt to draw a straight line between the two points from the right to the left.
(v) If at any point along this baseline, the actual curve is below the baseline then stop and move the right point one in along the curve.
(vi) Repeat steps (ii) to (v) until either the two points meet (i.e., no peak was found—typically if the line is horizontal or straight) or until no portion of the curve is below the baseline (i.e., a valid peak was found).

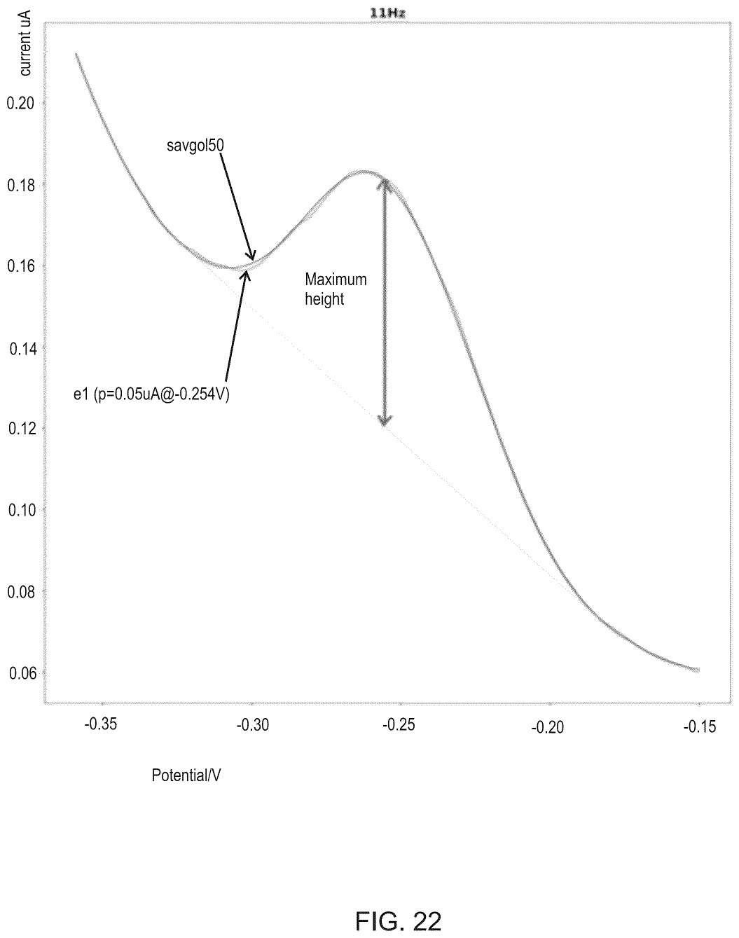

Once a baseline had been identified, finding the peak was simply the point at which the difference between the curve and that baseline is maximised as shown in FIG. 22.

The following parameters can be used to modify the behavior of the peak-finding algorithm. The values used for the clinical data are given in the “Current Setting” column.

| | | Current | |
|---|---|---|---|
| Parameter | Description | Setting | Impact |
| SAVGOL_POLYORDER | The order of the polynomial | 2 | A higher value will require |
| | function used to fit the window | | more computation and |
| | in the Savitzky-Golay filter. | | retain more variation |
| | | | (since it fits more complex |
| | | | curves to the sliding |
| | | | window). |
| SAVGOL_WINDOW_PCT | The percentage of the overall | 25 | A smaller window will |
| | data length that should be used | | retain more variation in |
| | for the Savitzky-Golay filter | | the data however too large |
| | window length. | | a value will over-simplify |
| | | | the curve. |

### Producing Analyte Concentration Responsive Signal S

The peak current magnitude from four different voltammograms was combined to produce the S values. The four voltammograms were produced using two different SWV frequencies, interrogating two different solutions.

The two frequencies were chosen so that they respond differently to the concentration of vancomycin in the solution, with one frequency giving a larger increase in peak current as vancomycin concentration increases and one giving either a smaller increase or a decrease in peak current as the vancomycin concentration increases. The purpose of using the two frequencies is to aid in correcting for underlying drift in the peak current magnitude due to non-analyte related effects such as electrode fouling and loss of active aptamer from the surface of the electrode over time. In this case 300 Hz was chosen as the more strongly increasing frequency and 50 Hz as the less strongly increasing frequency.

The peak currents measured in the presence of vancomycin were divided by the peak currents measured for the same sensing electrode in the absence of vancomycin. This calculates a peak current signal gain caused by the presence of vancomycin and was used to cancel out variation between electrodes in the exact amount of analyte responsive aptamers present on the electrode.

The formula used to calculate S for an individual electrode in these studies is:

$$S = \frac{{i}_{300}}{{i}_{300}^{0}} - \frac{{i}_{50}}{{i}_{50}^{0}}$$

where
i₃₀₀ the SWV peak current magnitude at 300 Hz in the test solution

$${i}_{300}^{0}$$

is the SWV peak current magnitude at 300 Hz in a solution with no vancomycin present
i₅₀ is the SWV peak current magnitude at 50 Hz in the test solution

$${i}_{50}^{0}$$

is the SWV peak current magnitude at 50 Hz in a solution with no vancomycin present.

For the clinical data, the zero vancomycin peak current values used in the calculations were the values measured between 15 and 45 minutes after application of the device, when the signal had initially stabilised and before any vancomycin was infused into the participant.

### Time Smoothing

The S values over time from the clinical data were then smoothed using a Savitzky-Golay filter with a 41 point sliding window fitted to a second order polynomial, with the points taken at 5 minute intervals. The smoothed S values were used from this point on.

### Producing the Calibration Function

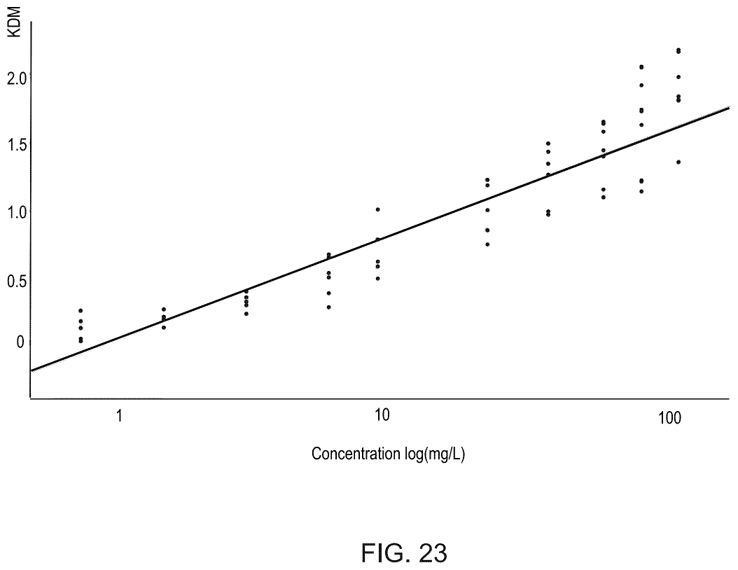

To produce a calibration function that converts S values into [V] values, three electrodes from the same production batch of electrodes used in the clinical experiments were tested in bovine plasma containing a range of vancomycin concentrations at 35° C. The plot shown at FIG. 23 is indicative of a calibration plot, where KDM corresponds to S values for multiple electrodes and the x-axis is the log of the known spiked concentration of vancomycin in the plasma.

Linear least squares was used to fit the S versus log (vancomycin concentration) data between 1 and 100 mg/L vancomycin concentration, to yield a straight line with Slope and an Intercept value. That is:

$$S = Slope \cdot log ( [ V ] ) + Intercept$$

The slope and intercept were used to convert S values from the clinical experiment into estimated vancomycin concentrations via the equation:

$$[ V ] = {10}^{( \frac{S - Intercept}{Slope} )}$$

It will be noted that the approach detailed above is a departure from conventional means implementing a binding isotherm equation would be used. The above approach was used for simplicity.

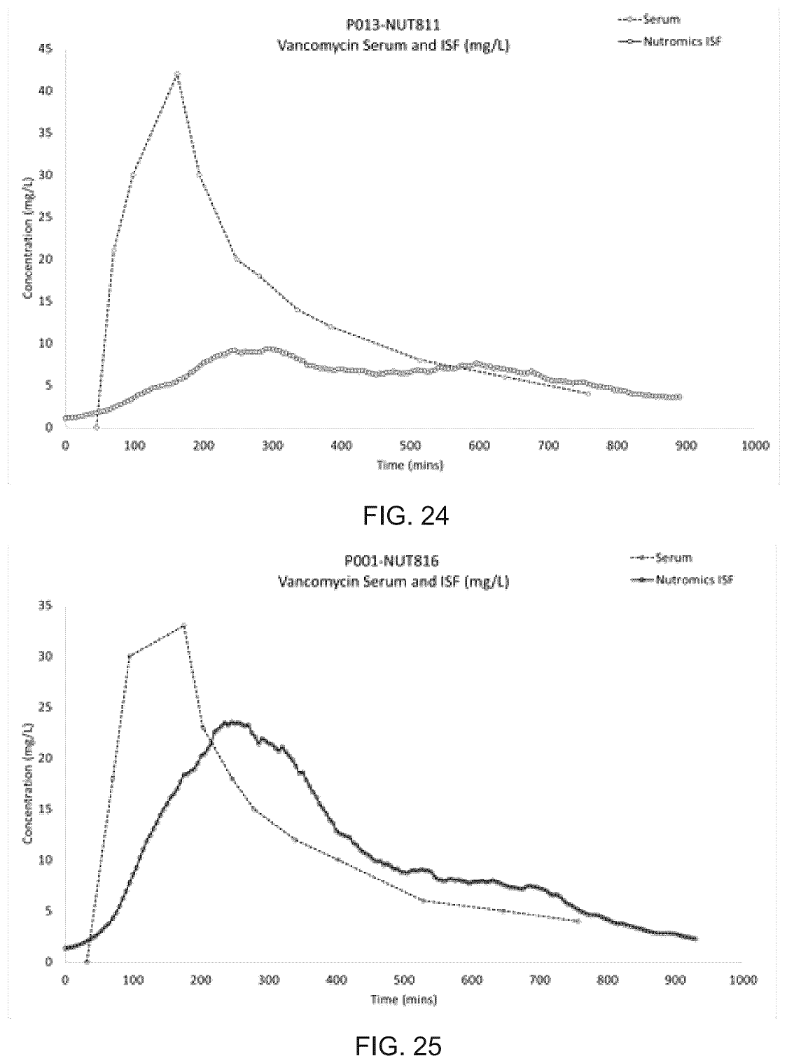

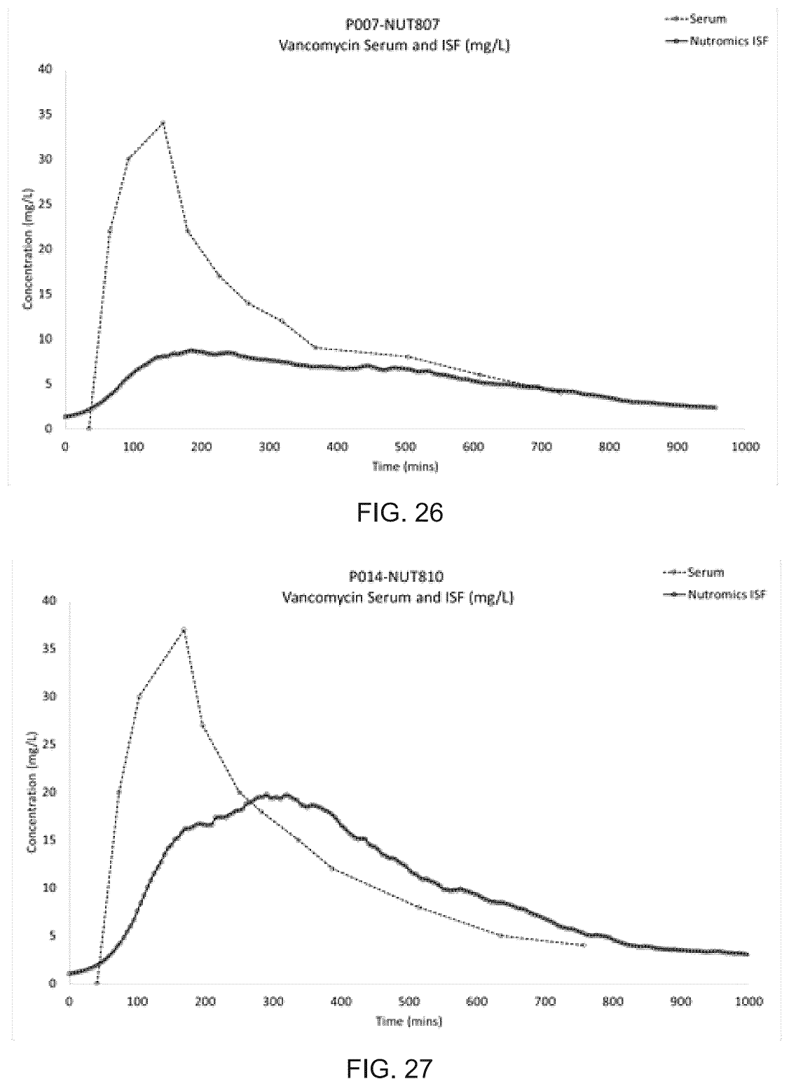

The average [V] value of the two electrodes in the device is that shown as the in vivo vancomycin concentration in the graphs of FIG. 24, FIG. 25, FIG. 26, and FIG. 27.

FIG. 24, FIG. 25, FIG. 26 and FIG. 27 show dynamics of vancomycin in ISF vs blood in the same participant. The ISF estimates are expressed in high resolution real-time monitoring that was measured every 5 minutes and reported instantly in real-time, such data being useful in personalising vancomycin therapy via a closed loop system. In this first in-human study all participants were screened to be healthy and yet high variation in vancomycin in the ISF compartment was observed. In some participants (FIG. 25 and FIG. 27), vancomycin perfused into the tissues to a much greater extent than for other participants (FIG. 24 and FIG. 26). Such variation establishes the need for personalised therapy to avoid excursions of a drug beyond a maximum toxic limit and a minimum efficacious limit.

Where ISF data shows a high level of drug perfusion into tissues, lower levels of drug would be administered, and vice versa, in the closed loop system such as that shown in FIG. 19. Such personalisation can be provided by way of the high resolution vancomycin data provided for the EAB sensor used in these studies. It is contemplated that high resolution data for any other drug that is detectable by an EAB sensor could be used in a closed loop system to maintain levels of the drug within a therapeutic window.

Conventional blood concentration data does not provide any further information in personalising therapy. Blood is only measured once every 24 hours in a clinical setting thereby limiting its use in personalisation and closed loop therapeutic drug administration systems. Even the measurements in this study (serially 12 times over a 12 hour period) is insufficient to properly personalise therapy.

Moreover, conventional blood measurements of a drug inform nothing as to the perfusion of a drug into tissues. Thus, the clinician has no information whatsoever as to the level of drug achieved in a target tissue, nor in the level in a tissue susceptible to toxicity (such as the kidneys or the liver). Information on drug levels in the relevant tissues (as distinct from the blood) provide significantly better control of a closed loop drug delivery system. Even with high resolution drug data from the blood, levels of the drug in relevant tissues will be ignored. The human studies described herein show for the first time that the concentration of drug in the tissues can be very different to that in the general circulation, and that appropriate dosage adjustment accounting for those differences can be made in a closed loop system.

Those skilled in the art will appreciate that the invention described herein is susceptible to further variations and modifications other than those specifically described. It is understood that the invention comprises all such variations and modifications which fall within the spirit and scope of the present invention.

While the invention has been disclosed in connection with the preferred embodiments shown and described in detail, various modifications and improvements thereon will become readily apparent to those skilled in the art. For example, the invention is described mainly by reference to the antibiotic vancomycin as the drug being monitored. The skilled person having the benefit of the present disclosure is enabled to apply the teachings herein to other drugs as referred to in the description and the following claims. For example, the substance may be therapeutic drug, including a cardiovascular drug, a respiratory drug, a gastrointestinal drug, a renal drug, a neurologic drug, a psychiatric drug, an endocrinology drug, a urologic drug, a rheumatologic drug, an ophthalmic drug, an otolaryngological drug, a dermatologic drug, an infectious disease drug, or a cancer drug.

Accordingly, the spirit and scope of the present invention is not to be limited by the foregoing examples, but is to be understood in the broadest sense allowable by law.

1. A computer-implemented method for administering a drug to a subject, the method comprising:
administering the drug to the subject using a processor-controllable agent administration apparatus,
contacting a fluid of the subject with an electrochemical aptamer-based sensor capable of detecting the drug,
receiving a series of output values of the electrochemical aptamer-based sensor over a period of time, and
using the series of output values or derivatives thereof to control the processor-controllable agent administration apparatus,
wherein the output values are received at a time interval or an averaged time interval of less than about 4 hours, 3 hours, 2 hours, 1 hour, 50 minutes, 40 minutes, 30 minutes, 20 minutes, 10 minutes, 5 minutes, 4 minutes, 3 minutes, 2 minutes, 1 minute, 50 seconds, 40 seconds, 30 seconds, 20 seconds, 10 seconds, 9 seconds, 8 seconds, 7 seconds, 6 seconds, 5 seconds, 4 seconds, 3 seconds, 2 seconds, 1 second, 900 milliseconds, 800 milliseconds, 700 milliseconds, 600 milliseconds, 500 milliseconds, 400 milliseconds, 300 milliseconds, 200 milliseconds, 100 milliseconds, 90 milliseconds, 80 milliseconds, 70 milliseconds, 60 milliseconds, 50 milliseconds, 40 milliseconds, 30 milliseconds, 20 milliseconds, 10 milliseconds, 9 milliseconds, 8 milliseconds, 7 milliseconds, 6 milliseconds, 5 milliseconds, 4 milliseconds, 3 milliseconds, 2 milliseconds, or 1 millisecond.

2. The method of claim 1, wherein the processor-controllable agent administration apparatus is a processor-controllable pump.

3. The method of claim 1, wherein the series of output values or derivatives thereof of the electrochemical aptamer-based sensor are used to generate a series of drug concentration values or derivatives thereof, and the processor-controllable agent delivery apparatus is controlled by reference to the series of drug concentration values or derivatives thereof.

4. The method of claim 3, wherein where the series of drug concentration values or derivatives thereof indicate that the subject has or may be about to receive a maximum or supra-maximum amount of the drug, the processor-controllable agent delivery apparatus is controlled so as to cease or reduce a rate of administration of the drug.

5. The method of claim 4, wherein indication of the maximum or supra-maximum amount is determined by reference to a concentration value for the drug as determined by a concentration value of the series of drug concentration values or derivatives thereof.

6. The method of claim 4, wherein the maximum amount is a toxic amount, a potentially toxic amount, or a wasteful amount.

7. The method of claim 3, wherein where the series of drug concentration values or derivatives thereof indicate that the subject has or may be about to receive a minimum or sub-minimum amount of the drug, the processor-controllable agent delivery apparatus is controlled so as to commence or increase a rate of administration of the drug.

8. The method of claim 7, wherein indication of the minimum or sub-minimum amount is determined by reference to a concentration value for the drug as determined by a concentration value of the series of drug concentration values or derivatives thereof.

9. The method of claim 7, wherein the minimum amount is a minimum therapeutically or prophylactically effective amount.

10. The method of claim 4, wherein indication that the subject has or may be about to receive a minimum or sub-minimum or maximum or supra-maximum amount of the drug is determined by reference to a kinetic parameter of the drug in the fluid.

11. The method of claim 10, wherein the kinetic parameter is a rate of increase or decrease in the amount of the drug.

12. The method of claim 4, wherein the series of output values or derivatives thereof of the electrochemical aptamer-based sensor are used to control the processor-controllable agent administration apparatus to maintain the amount of drug within a predetermined range.

13. An apparatus for controlled administration a drug to a subject, the apparatus comprising:
an electrochemical or derivatives thereof sensor configured to contact a fluid of the subject, and
a processor in operable communication with the electrochemical aptamer-based sensor and having access to processor-executable instructions configuring the processor to:
receive a series of output values of the electrochemical aptamer-based sensor over a period of time, and
the processor and/or other processor(s) having access to the processor-executable instructions using the series of output values or derivatives thereof to predict the pharmacokinetic feature,
wherein the output values or derivatives thereof are received at a time interval or an averaged time interval of less than about 4 hours, 3 hours, 2 hours, 1 hour, 50 minutes, 40 minutes, 30 minutes, 20 minutes, 10 minutes, 5 minutes, 4 minutes, 3 minutes, 2 minutes, 1 minute, 50 seconds, 40 seconds, 30 seconds, 20 seconds, 10 seconds, 9 seconds, 8 seconds, 7 seconds, 6 seconds, 5 seconds, 4 seconds, 3 seconds, 2 seconds, 1 second, 900 milliseconds, 800 milliseconds, 700 milliseconds, 600 milliseconds, 500 milliseconds, 400 milliseconds, 300 milliseconds, 200 milliseconds, 100 milliseconds, 90 milliseconds, 80 milliseconds, 70 milliseconds, 60 milliseconds, 50 milliseconds, 40 milliseconds, 30 milliseconds, 20 milliseconds, 10 milliseconds, 9 milliseconds, 8 milliseconds, 7 milliseconds, 6 milliseconds, 5 milliseconds, 4 milliseconds, 3 milliseconds, 2 milliseconds, or 1 millisecond.

14. The apparatus of claim 13, wherein the processor-controllable agent administration apparatus is a processor-controllable pump.

15. The apparatus of claim 13, wherein the series of output values or derivatives thereof of the electrochemical aptamer-based sensor are used to generate a series of drug concentration values, and the processor-controllable agent delivery apparatus is controlled by reference to the series of drug concentration values or derivatives thereof.

16. The apparatus of claim 15, wherein where the series of drug concentration values or derivatives thereof indicate that the subject has or may be about to receive a maximum or supra-maximum amount of the drug, the processor-controllable agent delivery apparatus is controlled so as to cease or reduce a rate of administration of the drug.

17. The apparatus of claim 15, wherein where the series of drug concentration values or derivatives thereof indicate that the subject has or may be about to receive a minimum or sub-minimum amount of the drug, the processor-controllable agent delivery apparatus is controlled so as to commence or increase a rate of administration of the drug.

18. The apparatus of claim 17, wherein indication of the minimum or sub-minimum amount is determined by reference to an exposure for the drug over a period of time, the exposure determined by reference to the series of drug concentration values or derivatives thereof.

19. The apparatus of claim 16, wherein indication that the subject has or may be about to receive a minimum or sub-minimum or maximum or supra-maximum amount of the drug is determined by reference to a kinetic parameter of the drug in the fluid, including a rate of increase or decrease in the amount of the drug.

20. The apparatus of claim 16, wherein the series of output values or derivatives thereof of the electrochemical aptamer-based sensor are used to control the processor-controllable agent administration apparatus to maintain the amount of drug within a predetermined range.
